(19) United States
(12) Patent Application Publication
(10) Pub. No.: US 2023/0200388 A1
(43) Pub. Date: Jun. 29, 2023

(54) COMPOUNDS AND METHODS FOR STIMULATING PLANTS

(71) Applicants: Travis S. BAYER, San Francisco, CA (US); Allison SCHWARTZ, Berkeley, CA (US); Christian IBARRA, Half Moon Bay, CA (US); Shailaja CHADHA, Emeryville, CA (US); Sound Agriculture Company, Emeryville, CA (US)

(72) Inventors: Travis S. BAYER, San Francisco, CA (US); Allison SCHWARTZ, Berkeley, CA (US); Christian IBARRA, Half Moon Bay, CA (US); Shailaja CHADHA, Emeryville, CA (US)

(73) Assignee: Sound Agriculture Company, Emeryville, CA (US)

(21) Appl. No.: 18/000,350

(22) PCT Filed: Jun. 3, 2021
(86) PCT No.: PCT/US2021/035770

§ 371 Date: Nov. 30, 2022

Related U.S. Application Data
(60) Provisional application No. 63/034,228, filed on Jun. 3, 2020.
(60) Provisional application No. 63/034,228, filed on Jun. 3, 2020.

(51) Int. Cl. A01N 43/16; A01N 63/22; A01P 21/00; A01N 25/02

(52) U.S. Cl. CPC A01N 43/16; A01N 63/22; A01P 21/00; A01N 25/02

(57) ABSTRACT

Disclosed herein are compounds or salts thereof, and compositions thereof, for increasing plant growth. Also disclosed are methods of increasing levels of plant nutrients using a compound, salt, or composition as disclosed herein. Also disclosed herein are kits comprising a compound, salt, or composition as described herein.

### CROSS-REFERENCE

This application claims benefit of U.S. Provisional Patent Application No. 63/034,228 filed on Jun. 3, 2020, incorporated herein by reference in its entirety.

### BRIEF SUMMARY

In one aspect, disclosed herein are liquid compositions that can comprise: (a) a compound or salt thereof of Formula I, Formula II, or Formula III:

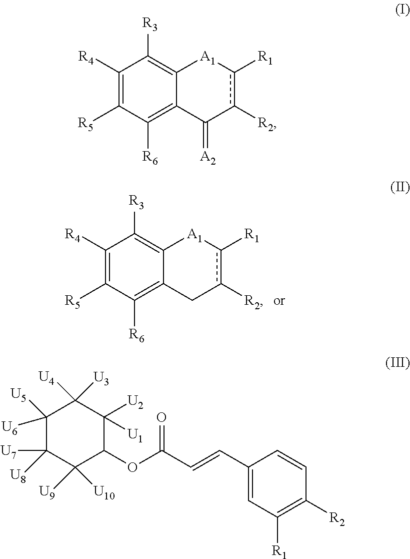

where: A₁ and A₂ can independently be O or S; R₁ and R₂ can independently be —H, —OH, —COOH, —SH, C₁-C₆ alkyl, C₃-C₆ cycloalkyl, or —Xₚ, where —Xₚ can be:

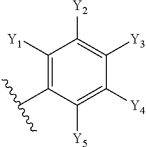

where Y₁, Y₂, Y₃, Y₄, and Y₅ can independently be —H, —OH, —SH, —F, —Cl, —Br, —I, or —O—Z₁, where Z₁ can be C₁-C₄ alkyl; or where R₁ and R₂ along with the carbon atoms connecting them can form a five or six-membered cycloalkyl ring or cycloalkenyl ring, or a five or six-membered aryl ring; U₁, U₂, U₃, U₄, U₅, U₆, U₇, U₈, U₉, and U₁₀ can independently be —H, —OH, —COOH, —SH, —F, —Cl, —Br, —I, —COO—Z₁, or —O—Z₁, wherein Z₁ is C₁-C₄ alkyl; R₃, R₄, R₅, and R₆ can independently be —H, —OH, —F, —Cl, —Br, —I, or —SH; and (b) an excipient, diluent, or carrier; where the liquid composition can comprise an amount of the compound or salt thereof that can at least partially stimulate: (a) an increased level of soluble orthophosphate of at least about 20% after contacting the amount of the compound or salt thereof with a live Bacillus megaterium bacteria strain, relative to a level of the soluble orthophosphate produced by a live Bacillus megaterium bacteria strain prior to the contacting, as determined by an in vitro assay that can comprise: (i) incubating a live Bacillus megaterium bacteria strain at an optical density at 600 nm (OD₆₀₀) of 0.02 with tricalcium phosphate at a final concentration of about 50 mM; (ii) collecting a sample of a liquid culture from a live Bacillus megaterium bacteria strain 72 hours after the incubating; and (iii) quantifying the level of the orthophosphate in the liquid culture using a malachite-green method; or (b) an increased level of nitrogen fixation after contacting the amount of the compound or salt thereof with a reporter Azotobacter vinelandii bacteria strain, relative to a level of the nitrogen fixation produced by a reporter Azotobacter vinelandii bacteria strain prior to the contacting, as determined by an in vitro assay that can comprise: (i) incubating a reporter Azotobacter vinelandii bacteria strain aerobically in nitrogen-free media at an OD₆₀₀ of 0.02, wherein a reporter Azotobacter vinelandii bacteria strain is transformed with a luciferase reporter plasmid configured to produce a higher level luminescence in response to nitrogen fixation; (ii) contacting a reporter Azotobacter vinelandii bacteria strain with luciferin 24 hours after the incubating; and (iii) quantifying the level of the luminescence using a luminometer, where a higher level of luminescence can correspond to a higher degree of nitrogen fixation by a reporter Azotobacter vinelandii bacteria strain; or (c) any combination thereof.

In another aspect, disclosed herein are liquid compositions that can comprise: (a) a compound or salt thereof of Formula I, Formula II, or Formula III:

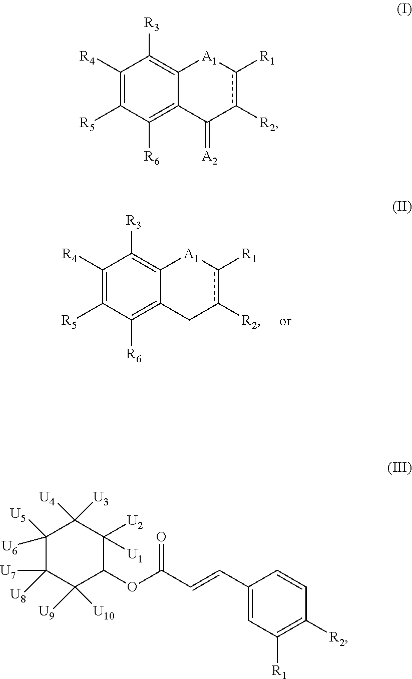

where: A₁ and A₂ can independently be O or S; R₁ and R₂ can independently be —H, —OH, COOH, —SH, C₁-C₆ alkyl, C₃-C₆ cycloalkyl, or —Xₚ, where —Xₚ can be:

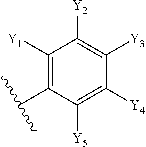

where Y₁, Y₂, Y₃, Y₄, and Y₅ can independently be —H, —OH, —SH, —F, —Cl, —Br, —I, or —O—Z₁, where Z₁ can be C₁-C₄ alkyl; or where R₁ and R₂ along with the carbon atoms connecting them can form a five or six-membered cycloalkyl ring or cycloalkenyl ring, or a five or six-membered aryl ring; U₁, U₂, U₃, U₄, U₅, U₆, U₇, U₈, U₉, and U₁₀ can independently be —H, —OH, —COOH, —SH, —F, —Cl, —Br, —I, —COO—Z₁, or —O—Z₁, wherein Z₁ is C₁-C₄ alkyl; R₃, R₄, R₅, and R₆ can independently be —H, —OH, —F, —Cl, —Br, —I, or —SH; and (b) an excipient, diluent, or carrier; where the compound or salt thereof is present in the composition at a concentration of from about 0.1 μM to 30 μM.

In another aspect, disclosed herein are liquid compositions that can comprise: (a) a compound or salt thereof of Formula I, Formula II, or Formula III:

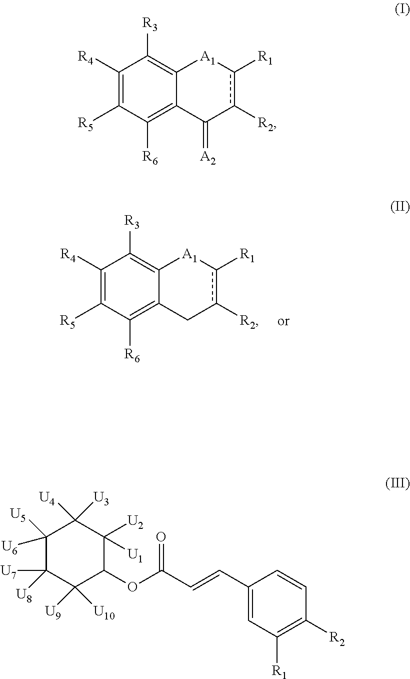

where: A₁ and A₂ can independently be O or S; R₁ and R₂ can independently be —H, —OH, COOH, —SH, C₁-C₆ alkyl, C₃-C₆ cycloalkyl, or —Xₚ, where —Xₚ can be:

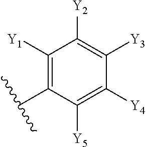

where Y₁, Y₂, Y₃, Y₄, and Y₅ can independently be —H, —OH, —SH, —F, —Cl, —Br, —I, or —O—Z₁, where Z₁ can be C₁-C₄ alkyl; or where R₁ and R₂ along with the carbon atoms connecting them can form a five or six-membered cycloalkyl ring or cycloalkenyl ring, or a five or six-membered aryl ring; U₁, U₂, U₃, U₄, U₅, U₆, U₇, U₈, U₉, and U₁₀ can independently be —H, —OH, —COOH, —SH, —F, —Cl, —Br, —I, —COO—Z₁, or —O—Z₁, wherein Z₁ is C₁-C₄ alkyl; R₃, R₄, R₅, and R₆ can independently be —H, —OH, —F, —Cl, —Br, —I, or —SH; and (b) an excipient, diluent, or carrier; wherein the liquid composition comprises an amount of the compound or salt thereof that is at least partially effective to produce: (a) an increased level of soluble orthophosphate of at least about 20% after contacting the amount of the compound or salt thereof with a live microbe, relative to a level of the soluble orthophosphate produced by the live microbe prior to the contacting, as determined by an in vitro assay comprising: (i) incubating the live microbe at an optical density at 600 nm (OD₆₀₀) of 0.02 with tricalcium phosphate at a final concentration of about 50 mM; (ii) collecting a sample of a liquid culture from the live microbe 72 hours after the incubating; and (iii) quantifying the level of the orthophosphate in the liquid culture using a malachite-green method; or (b) an increased level of nitrogen fixation after contacting the amount of the compound or salt thereof with the live microbe, relative to a level of the nitrogen fixation produced by the live microbe prior to the contacting, as determined by an in vitro assay comprising: (i) incubating the live microbe aerobically in nitrogen-free media at an OD₆₀₀ of 0.02, wherein the live microbe is transformed with a luciferase reporter plasmid configured to produce a higher level luminescence in response to nitrogen fixation; (ii) contacting the live microbe with luciferin 24 hours after the incubating; and (iii) quantifying the level of the luminescence using a luminometer, wherein a higher level of luminescence corresponds to a higher degree of nitrogen fixation by the live microbe; or any combination thereof. In some embodiments, a compound or its salt can be present at a concentration of from about 0.1 μM to about 20 μM. In some embodiments, a composition can comprise a diluent. In some embodiments, a diluent can be agriculturally acceptable. In some embodiments, a diluent can comprise a plant oil. In some embodiments, a plant oil can be selected from the group consisting of sunflower oil, canola oil, avocado seed oil, grapeseed oil, almond oil, cocoa butter, coconut oil, corn oil, cottonseed oil, flax seed oil, hemp oil, olive oil, palm kernel oil, peanut oil, pumpkin seed oil, rice bran oil, safflower oil, sesame seed oil, soybean oil, walnut oil, and any combination thereof. In some embodiments, a liquid composition that comprises a compound or salt thereof can be of Formula Ia, Ib, Ic, or Id:

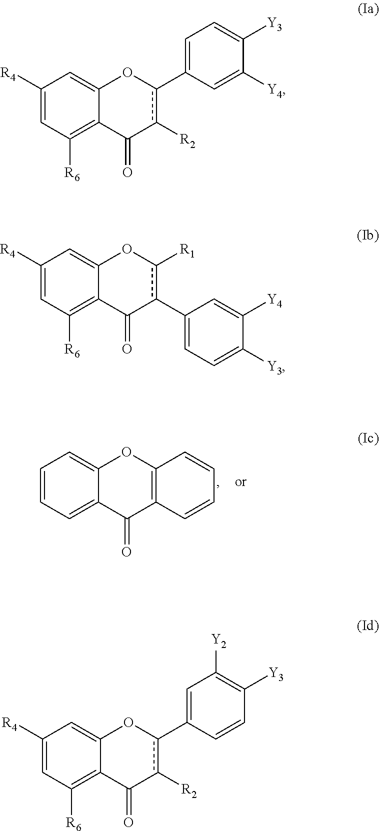

where R₁, R₂, R₄, R₆, Y₂, Y₃, and Y₄ are as defined above. In some embodiments, a compound or salt thereof can be of Formula Ia. In some embodiments, a compound can be selected from the group consisting of:

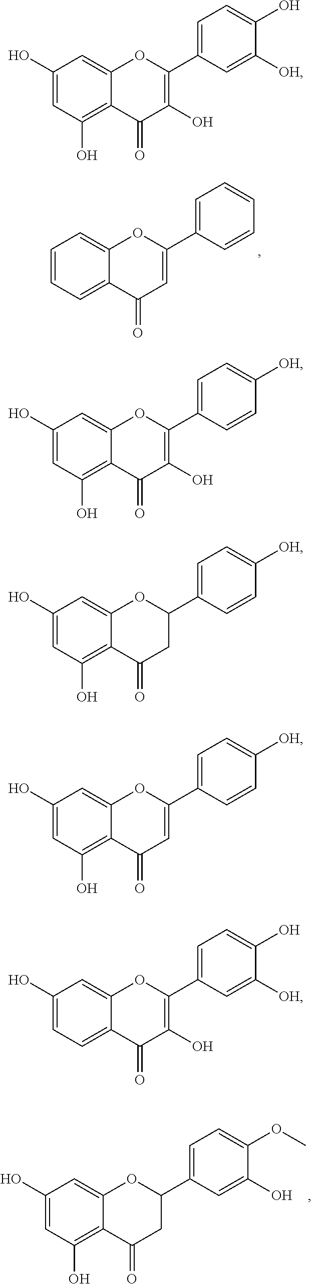

or a salt of any of these. In some embodiments, a compound or salt thereof can be of Formula Ib. In some embodiments, a compound can be selected from the group consisting of:

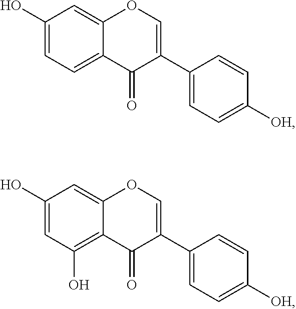 [SMILES: O=c1c(-c2ccc(O)cc2)coc2cc(O)ccc12 ; O=c1c(-c2ccc(O)cc2)coc2cc(O)cc(O)c12]

or a salt of any of these. In some embodiments, a compound or salt thereof can be of Formula Ic or a salt thereof. In some embodiments, a compound or salt thereof can be of Formula Id. In some embodiments, a compound or a salt thereof is of Formula Id is:

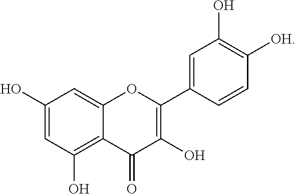 [SMILES: O=c1c(O)c(-c2ccc(O)c(O)c2)oc2cc(O)cc(O)c12]

In some embodiments, a compound or salt thereof can be of Formula IIa:

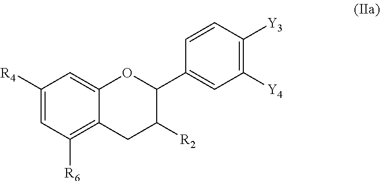

where R₂, R₄, R₆, Y₃, and Y₄ are as defined above. In some embodiments, a compound or salt thereof can be selected from the group consisting of:

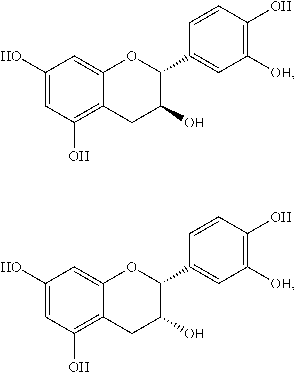 [SMILES: Oc1cc(O)c2c(c1)O[C@H](c1ccc(O)c(O)c1)[C@@H](O)C2 ; Oc1cc(O)c2c(c1)O[C@H](c1ccc(O)c(O)c1)[C@H](O)C2]

or

a salt of either of these. In some embodiments, a compound or salt thereof can be of Formula IIIa:

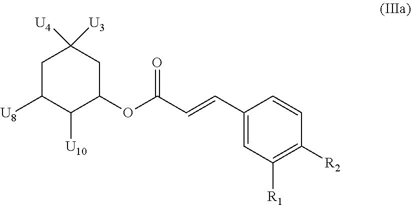

where R₁, R₂, U₃, U₄, U₈, and U₁₀ are as defined above. In some embodiments, a compound or salt thereof can be:

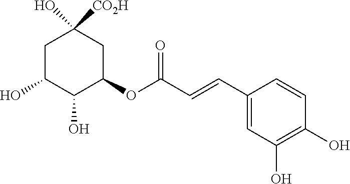 [SMILES: O=C(/C=C/c1ccc(O)c(O)c1)O[C@@H]1C[C@](O)(C(=O)O)C[C@@H](O)[C@H]1O]

or a salt thereof. In some embodiments, the live microbe is present in soil. In some embodiments, the live microbe is a bacteria strain, an actinomycete, a fungus, a protozoa, or any combination thereof. In some embodiments, the live microbe is a bacteria strain of genus Bacillus, Azobacter, Pseudomonas, Nitrobacter, Clostrodium, or any combination thereof. In some embodiments, the live microbe is selected from the group consisting of: Azotobacter chroococcum, Pseudomonas stutzeri, Pseudomonas pseudoalcaligenes, Massilia tieshanesis, Massilia aerilata, Massilia putida, Bacillus solisilvae, Bacillus niacini, Massilia agilis, Bacillus wiedmannii, Massilia brevitalea, Bacillus acidiceler, Bacillus toyonensis, Pseudomonas otitidis, Pseudomonas citronellolis, Paenibacillus qinlingensis, Massilia solisilvae, Massilia terrae, Bacillus paramycoides, Massilia aurea, Bacillus acidicola, Panenibacillus alginolyticus, Bacillus novalis, Pseudomonas aeruginosa, Bacillus halmapalus, Pseudomonas knackmussii, Klebsiella pneumoniae, Klebsiella variicola, Klebsiella oxytoca, Pseudomonas aeruginosa, Serratia marcescens, Bacillus amyloliquefaciens, Gluconacetobacter diazotrophicus Massilia arvi, Massilia agri, Massilia pinisoli, Bacillus megaterium, Bacillus bataviensis, Massilia chloroacetimidivorans, Bacillus mycoides, Bacillus flexus, Bacillus simplex, Pseudomonas balearica, Pseudomonas plecoglossicida, Caballeronia turbans, Psychobacillus lasiicaptis, Bacillus soli, Bacillus cohnii, Cupriavidus campinensis, Brevibacterium frigoritolerans, Bacillus pocheonensis, Pseudomonas monteilii, Bacillus vireti, Bacillus pacificus, Paenibacillus taihuensis, Azotobacter beijerinckii, Paenibacillus contaminans, Bacillus drentensis, Bacillus thuringiensis, Bacillus firmus, Bacillus cereus, Bacillus mobilis, Bacillus luciferensis, Massilia niastensis, Bacillus cucumis, Pseudomonas flavescens, Massilia timonae, Massilia kyonggiensis, Pseudomonas indica, Bacillus phyllosphaerae, Pseudomonas guguanensis, Paenibacillus beijingensis, Bacillus pseudomycoides, Adhaeribacter terreus, Microvirga zambiensis, Pseudomonas oryzae, or any combination thereof.

Also disclosed herein are methods that can comprise contacting a composition with a live microbe. In some embodiments, a composition can comprise: (a) a compound or salt thereof of Formula I, Formula II, or Formula III:

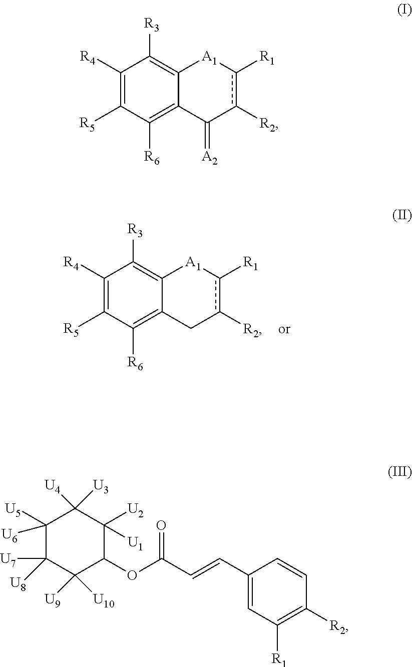

where: A₁ and A₂ can independently be O or S; R₁ and R₂ can independently be —H, —OH, —COOH, —SH, C₁-C₆ alkyl, C₃-C₆ cycloalkyl, or —Xₚ, where —Xₚ can be:

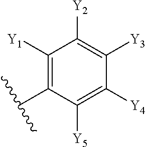

where Y₁, Y₂, Y₃, Y₄, and Y₅ can independently be —H, —OH, —SH, —F, —Cl, —Br, —I, or —O—Z₁, where Z₁ can be C₁-C₄ alkyl, or where R₁ and R₂ along with the carbon atoms connecting them can form a five or six-membered cycloalkyl ring or cycloalkenyl ring, or a five or six-membered aryl ring; U₁, U₂, U₃, U₄, U₅, U₆, U₇, U₈, U₉, and U₁₀ can independently be —H, —OH, —COOH, —SH, —F, —Cl, —Br, —I, —COO—Z₁, or —O—Z₁, wherein Z₁ can be C₁-C₄ alkyl, and R₃, R₄, R₅, and R₆ can independently be —H, —OH, —F, —Cl, —Br, —I, or —SH; and (b) an excipient, diluent, or carrier; where the contacting can be sufficient to produce: (a) an increased level of soluble orthophosphate of at least about 20% after contacting the amount of the compound or salt thereof with the live microbe, relative to a level of the soluble orthophosphate produced by the live microbe prior to the contacting, as determined by an in vitro assay that can comprise: (i) incubating a live Bacillus megaterium bacteria strain at an optical density at 600 nm (OD₆₀₀) of 0.02 with tricalcium phosphate at a final concentration of about 50 mM; (ii) collecting a sample of a liquid culture from the live Bacillus megaterium bacteria strain 72 hours after the incubating; and (iii) quantifying the level of the orthophosphate in the liquid culture using a malachite-green method; or (b) an increased level of nitrogen fixation after contacting the amount of the compound or salt thereof with the live microbe, relative to a level of the nitrogen fixation produced by the live microbe prior to the contacting, as determined by an in vitro assay that can comprise: (i) incubating a reporter Azotobacter vinelandii bacteria strain aerobically in nitrogen-free media at an OD₆₀₀ of 0.02, wherein the reporter Azotobacter vinelandii bacteria strain is transformed with a luciferase reporter plasmid configured to produce a higher level luminescence in response to nitrogen fixation; (ii) contacting the reporter Azotobacter vinelandii bacteria strain with luciferin 24 hours after the incubating; and (iii) quantifying the level of the luminescence using a luminometer, wherein a higher level of luminescence corresponds to a higher degree of nitrogen fixation by the reporter Azotobacter vinelandii bacteria strain; or (c) any combination thereof. In some embodiments, a composition can comprise a diluent. In some embodiments, a diluent can be agriculturally acceptable. In some embodiments, a diluent can comprise a plant oil. In some embodiments, a plant oil can be selected from the group consisting of sunflower oil, canola oil, avocado seed oil, grapeseed oil, almond oil, cocoa butter, coconut oil, corn oil, cottonseed oil, flax seed oil, hemp oil, olive oil, palm kernel oil, peanut oil, pumpkin seed oil, rice bran oil, safflower oil, sesame seed oil, soybean oil, walnut oil, and any combination thereof. In some embodiments, a compound or salt thereof can be of Formula Ia, Ib, Ic, or Id:

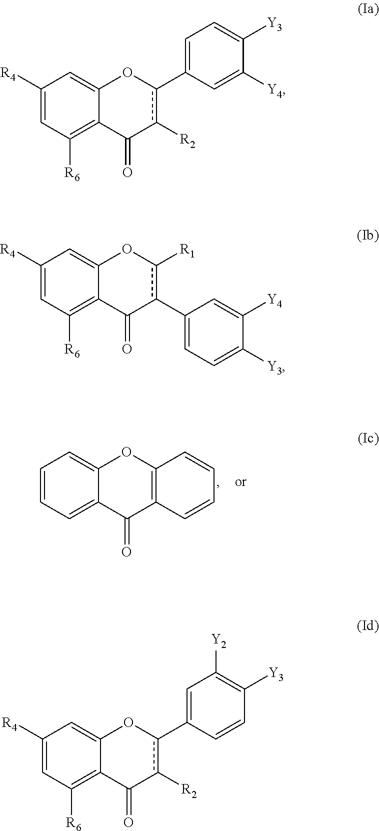

where R₁, R₂, R₄, R₆, Y₂, Y₃, and Y₄ are as defined above. In some embodiments, a compound or salt thereof can be of Formula Ia. In some embodiments, a compound can be selected from the group consisting of:

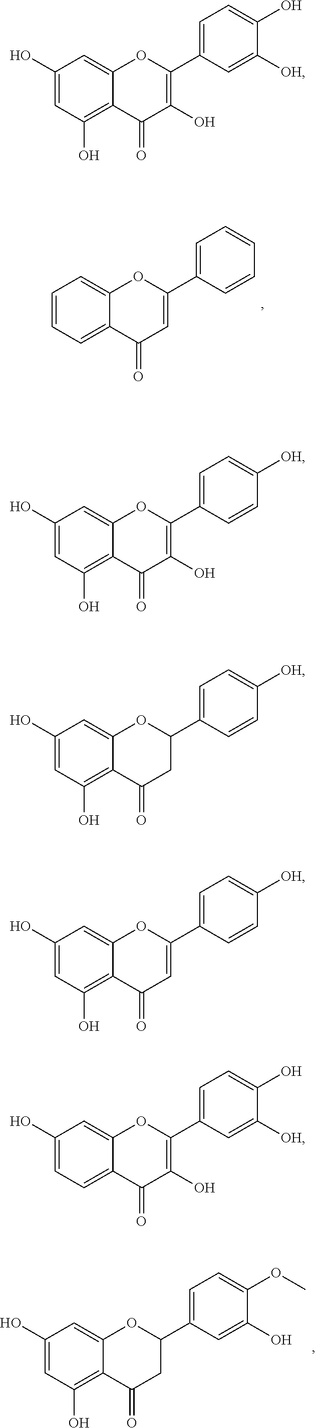

or a salt of any of these. In some embodiments, a compound or salt thereof can be of Formula Ib. In some embodiments, a compound can be selected from the group consisting of:

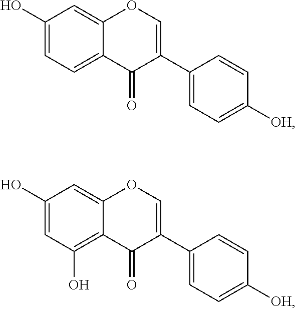 [SMILES: O=c1c(-c2ccc(O)cc2)coc2cc(O)ccc12 ; O=c1c(-c2ccc(O)cc2)coc2cc(O)cc(O)c12]

or a salt of any of these. In some embodiments, a compound or salt thereof can be of Formula Ic or a salt thereof. In some embodiments, a compound or salt thereof can be of Formula Id. In some embodiments, a compound or a salt thereof is of Formula Id:

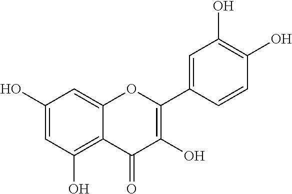 [SMILES: O=c1c(O)c(-c2ccc(O)c(O)c2)oc2cc(O)cc(O)c12]

In some embodiments, a compound or salt thereof can be of Formula IIa:

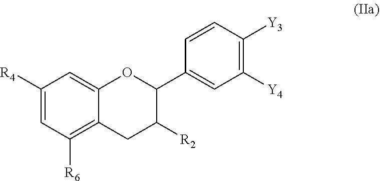

where R₂, R₄, R₆, Y₃, and Y₄ are as defined above. In some embodiments, a compound or salt thereof can be selected from the group consisting of:

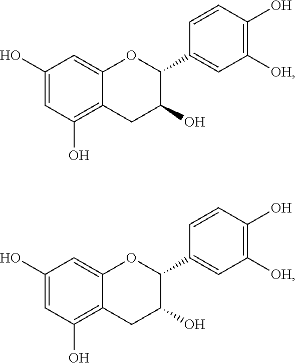 [SMILES: Oc1cc(O)c2c(c1)O[C@H](c1ccc(O)c(O)c1)[C@@H](O)C2 ; Oc1cc(O)c2c(c1)O[C@H](c1ccc(O)c(O)c1)[C@H](O)C2]

or a salt of either of these. In some embodiments, a compound or salt thereof can be of Formula IIIa:

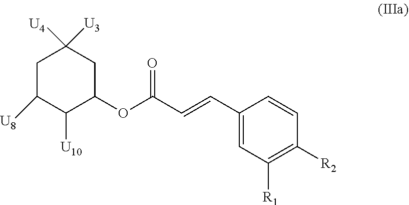

where R₁, R₂, U₃, U₄, U₈, and U₁₀ are as defined above. In some embodiments, a compound or salt thereof can be:

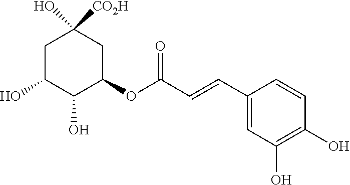 [SMILES: O=C(/C=C/c1ccc(O)c(O)c1)O[C@@H]1C[C@](O)(C(=O)O)C[C@@H](O)[C@H]1O]

or a salt thereof. In some embodiments, a live microbe can be present in soil. In some embodiments, a live microbe can be a bacteria strain, an actinomycete, a fungus, a protozoa, or any combination thereof. In some embodiments, a live microbe can be a bacteria strain of genus Bacillus, Azobacter. Pseudomonas, Nitrobacter, Clostrodium, or any combination thereof. In some embodiments, a live microbe can be selected from the group consisting of: Azotobacter chroococcum, Pseudomonas stutzeri, Pseudomonas pseudoalcaligenes, Massilia tieshanesis, Massilia aerilata, Massilia putida, Bacillus solisilvae, Bacillus niacini, Massilia agilis, Bacillus wiedmannii, Massilia brevitalea, Bacillus acidiceler, Bacillus toyonensis, Pseudomonas otitidis, Pseudomonas citronellolis, Paenibacillus qinlingensis, Massilia solisilvae, Massilia terrae, Bacillus paramycoides, Massilia aurea, Bacillus acidicola, Panenibacillus alginolyticus, Bacillus novalis, Pseudomonas aeruginosa, Bacillus halmapalus, Pseudomonas knackmussii, Klebsiella pneumoniae, Klebsiella variicola, Klebsiella oxytoca, Pseudomonas aeruginosa, Serratia marcescens, Bacillus amyloliquefaciens, Gluconacetobacter diazotrophicus Massilia arvi, Massilia agri, Massilia pinisoli, Bacillus megaterium, Bacillus bataviensis, Massilia chloroacetimidivorans, Bacillus mycoides, Bacillus flexus, Bacillus simplex, Pseudomonas balearica, Pseudomonas plecoglossicida, Caballeronia turbans, Psychobacillus lasiicaptis, Bacillus soli, Bacillus cohnii, Cupriavidus campinensis, Brevibacterium frigoritolerans, Bacillus pocheonensis, Pseudomonas monteilii, Bacillus vireti, Bacillus pacificus, Paenibacillus taihuensis, Azotobacter beijerinckii, Paenibacillus contaminans, Bacillus drentensis, Bacillus thuringiensis, Bacillus firmus, Bacillus cereus, Bacillus mobilis, Bacillus luciferensis, Massilia niastensis, Bacillus cucumis, Pseudomonas flavescens, Massilia timonae, Massilia kyonggiensis, Pseudomonas indica, Bacillus phyllosphaerae, Pseudomonas guguanensis, Paenibacillus beijingensis, Bacillus pseudomycoides, Adhaeribacter terreus, Microvirga zambiensis, Pseudomonas oryzae, or any combination thereof. In some embodiments, a contacting can be performed at least about 1, 2, 3, 4, 5, or 6 times in a 24 hour time period. In some embodiments, a contacting can be performed at least about 1, 2, 3, 4, 5, 6, or 7 times in a week.

Also disclosed herein are methods of improving health of a plant. A method can comprise contacting a plant present in soil that can comprise a live microbe with a composition described herein. In some embodiments, a contacting can be sufficient to increase a biomass of a plant or an amount of greenness of a plant, relative to a biomass or amount of greenness of a comparable plant grown for a comparable amount of time and not contacted with the composition, thereby improving the health of a plant. In some embodiments, a contacting can comprise contacting a leaf of a plant. In some embodiments, a contacting can comprise contacting a stem of a plant. In some embodiments, a contacting can comprise contacting a root of a plant. In some embodiments, a contacting can substantially maintain an amount of greenness of a plant for a longer period of time, relative to an amount of greenness of a comparable plant.

Also disclosed herein are methods of making a plant. A method can comprise: (a) contacting a plant seed with an exogenous compound or salt thereof of Formula I, Formula II, or Formula III:

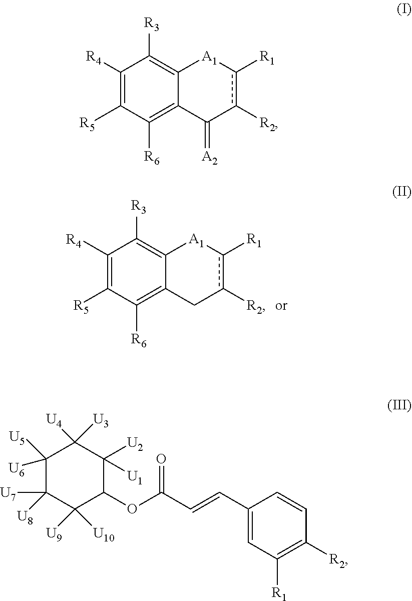

where: A₁ and A₂ can independently be O or S; R₁ and R₂ can independently be —H, —OH, —COOH, —SH, C₁-C₆ alkyl, C₃-C₆ cycloalkyl, or —Xₚ, where —Xₚ can be:

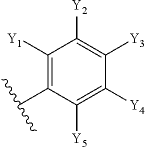

where Y₁, Y₂, Y₃, Y₄, and Y₅ can independently be —H, —OH, —SH, —F, —Cl, —Br, —I, or —O—Z₁, where Z₁ can be C₁-C₄ alkyl, or where R₁ and R₂ along with the carbon atoms connecting them can form a five or six-membered cycloalkyl ring or cycloalkenyl ring, or a five or six-membered aryl ring; U₁, U₂, U₃, U₄, U₅, U₆, U₇, U₈, U₉, and U₁₀ can independently be —H, —OH, —COOH, —SH, —F, —Cl, —Br, —I, —COO—Z₁, or —O—Z₁, wherein Z₁ can be C₁-C₄ alkyl, and R₃, R₄, R₅, and R₆ can independently be —H, —OH, —F, —Cl, —Br, —I, or —SH; and planting the plant seed into soil comprising a live microbe, thereby making a plant. In some embodiments, a contacting can be sufficient to increase a biomass of a plant, relative to a biomass of a comparable plant produced from a seed not contacted with a composition and grown for a comparable time. In some embodiments, a contacting can be sufficient to increase an amount of greenness of a plant, relative to an amount of greenness of a comparable plant produced from a seed not contacted with a composition and grown for a comparable time. In some embodiments, a compound or salt thereof can be of Formula Ia, Ib, Ic, or Id:

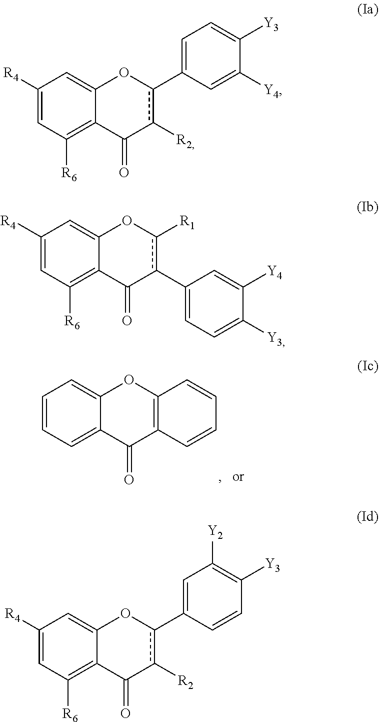

where R₁, R₂, R₄, R₆, Y₂, Y₃, and Y₄ are as defined above. In some embodiments, a compound or salt thereof can be of Formula Ia. In some embodiments, a compound can be selected from the group consisting of:

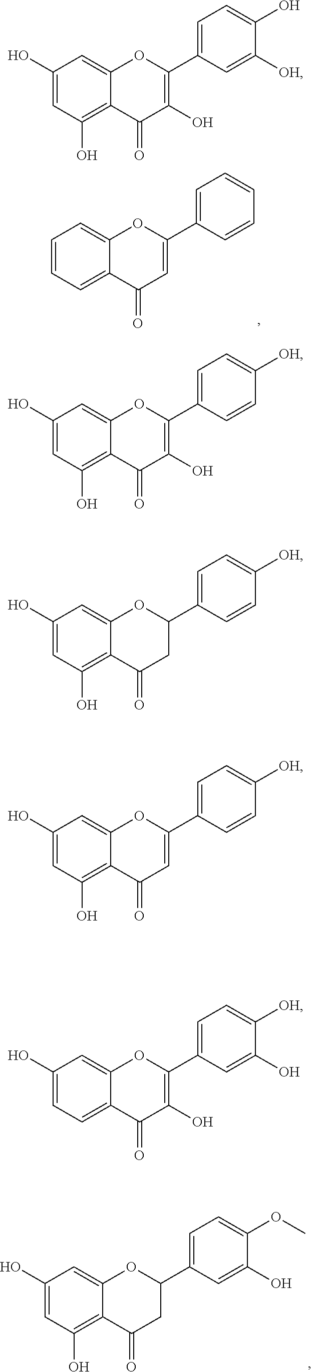

or a salt of any of these. In some embodiments, a compound or salt thereof can be of Formula Ib. In some embodiments, a compound can be selected from the group consisting of:

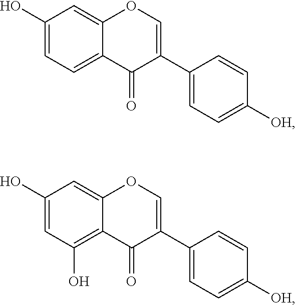 [SMILES: O=c1c(-c2ccc(O)cc2)coc2cc(O)ccc12 ; O=c1c(-c2ccc(O)cc2)coc2cc(O)cc(O)c12]

or a salt of any of these. In some embodiments, a compound or salt thereof can be of Formula Ic or a salt thereof. In some embodiments, a compound or salt thereof can be of Formula Id. In some embodiments, a compound of Formula Id is:

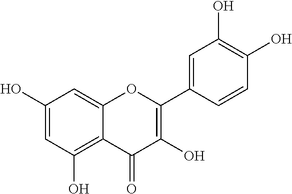 [SMILES: O=c1c(O)c(-c2ccc(O)c(O)c2)oc2cc(O)cc(O)c12]

or a salt thereof. In some embodiments, a compound or salt thereof can be of Formula IIa:

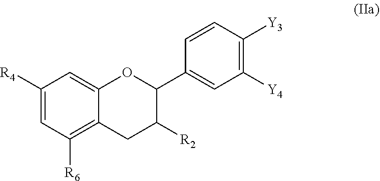

where R₂, R₄, R₆, Y₃, and Y₄ are as defined above. In some embodiments, a compound or salt thereof can be selected from the group consisting of:

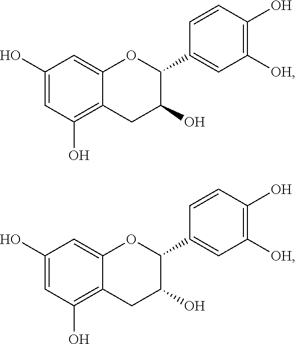 [SMILES: Oc1cc(O)c2c(c1)O[C@H](c1ccc(O)c(O)c1)[C@@H](O)C2 ; Oc1cc(O)c2c(c1)O[C@H](c1ccc(O)c(O)c1)[C@H](O)C2]

or

a salt of either of these. In some embodiments, a compound or salt thereof can be of Formula IIIa:

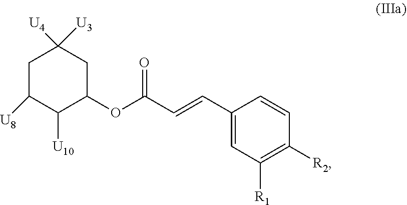

where R₁, R₂, U₃, U₄, U₈, and U₁₀ are as defined above. In some embodiments, a compound or salt thereof can be:

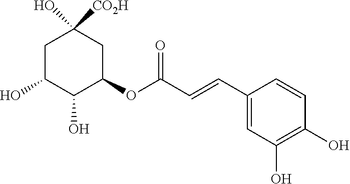 [SMILES: O=C(/C=C/c1ccc(O)c(O)c1)O[C@@H]1C[C@](O)(C(=O)O)C[C@@H](O)[C@H]1O]

or a salt thereof

Also disclosed herein are isolated plant seeds that can comprise a liquid composition as described herein.

Also disclosed herein are kits that can comprise a liquid composition as described herein in a container. In some embodiments, a container can be a spray bottle, a syringe, a vial, or a bucket.

Also disclosed herein are kits that can comprise an isolated plant seed as described herein in a container. In some embodiments, a container can be a pouch. In some embodiments, a kit can further comprise soil, fertilizer, or a combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of exemplary embodiments are set forth with particularity in the appended claims. A better understanding of the features and advantages will be obtained by reference to the following detailed description that sets forth illustrative embodiments, in which the principles of exemplary embodiments are utilized, and the accompanying drawings of which:

FIG. 1 depicts stimulation of phosphate solubilization in a model bacteria strain using an exemplary compound as described herein.

FIG. 2 depicts stimulation of phosphate solubilization in soil consortia using an exemplary compound as described herein.

FIG. 3 depicts induction of nitrogen fixing gene cluster in a reporter bacteria strain after contacting the bacteria strain with an exemplary compound as described herein.

FIG. 4 depicts an increase in plant biomass after contacting plants with compounds as described herein. Contacting the plants produced a significant increase in plant biomass, relative to plants not contacted with the compounds.

FIG. 5 displays phosphate solubilization activity induced by Formula Id in Bacillus megaterium. Phosphate levels were measured at 4 days post treatment. Two stars (**) represents a significant difference at p<0.01. Three technical replicates from each supernatant were tested. Orthophosphate was measured using the malachite-green phosphate method.

FIG. 6 shows activation of nifHₚᵣₒ::luciferase bioreporter by Formula Id. The nifHₚᵣₒ::luciferase bioreporter was activated the over controls, indicating an increase in nitrogenase gene expression in Azotobacter vinelandii, a free-living nitrogen fixing bacteria.

FIG. 7 depicts stimulation of phosphate solubilization by Formula Id. It was stimulated when applied as a spray to plant foliage. B73 Corn plants were grown until V3 growth stage, removed from potting soil, rinsed, and placed in tap water for 1.5 weeks to induce nutrient stress. Plants received foliar (3 mL/plant using a fingertip sprayer) applied treatments and were placed in 250 mL baffled flasks containing 50 mL NBRIP growth medium ([53 mM] Ca₃(PO₄)₂) and 500 mg of 2 mm particle-sized field soil. Flasks with treated corn and sterilized foam caps were placed on orbital shakers at 100 RPM for 1 day at room temperature under fluorescent lights. Orthophosphate was measured using the malachite-green phosphate method.

### DETAILED DESCRIPTION

Disclosed herein are compounds, salts thereof, and compositions containing a compound or salt thereof for increasing production of a nutrient (e.g. soluble orthophosphate or nitrogen) available to a plant. Also disclosed herein are methods of using a compound, salt, or composition as described herein to increase production of a nutrient available to a plant. Also disclosed herein are kits that can comprise a compound, salt, or composition as described herein in a container.

In some aspects, disclosed herein are small molecule compounds (e.g., molecular weight less than 500 daltons) that can serve as universal signals to native microbes to increase agronomically important activities is a potential solution for a standardized method to improve the soil's ability to provide nutrients to plants. In some instances, the small molecule can be a flavonoid. In some instances, a small molecule can act as a soil amendment and stimulate microbial phosphate solubilization and nitrogen fixation activity. In some instances, a small molecule can cause an increase in bacterial phosphate solubilization and nitrogen fixation in model microbial systems and in diverse consortia of soil microbes. In some instances, the small molecule can improve plant growth across several metrics. In some instances, the small molecule can enhance microbial activities known to be beneficial to plants and be an avenue toward the chemical reprogramming of the soil microbiome for improved plant health.

In some instances, an amount of orthophosphate can be determined in liquid cultures of a reporter bacteria strain (such as Bacillus megaterium), with and without the addition of a compound, salt, or formulation as described herein. At 72 hours, the average concentration of orthophosphate significantly increased due to contacting with a compound, salt, or formulation, as compared to control culture that was not contacted with a compound, salt, or formulation.

In some instances, an amount of nitrogen fixation can be determined in liquid cultures of a reporter bacteria strain, with and without the addition of a compound, salt, or formulation as described herein. A reporter bacteria strain can comprise a luciferase reporter gene that, when contacted with luciferin, can produce luminescence that can be proportional to an amount of nitrogen fixation. At 24 hours, an amount of nitrogen fixation can be significantly increased due to contacting with a compound, salt, or formulation, as compared to control culture that was not contacted with a compound, salt, or formulation.

In some instances, a compound, composition, method, or kit disclosed herein can release nutrients bound in soil to make them available for plant growth and enhance inoculant activity as well as the activity of endogenous soil microbes. Such enhanced plant nutrition leads to higher yield potential.

In some instances, a compound, composition, method, or kit disclosed herein can boost plants to release a signaling compound requiring nutrients (nitrogen and phosphorus) to soil microbes. Arbuscular mycorrhizal fungi (AMF) and phosphate solubilizing microbes (PSM) can sense these signals and increase phosphate solubilization and root symbiosis. As a result, nitrogen and phosphorous are liberated from the soil and available for uptake by plants.

In some instances, a compound, composition, method, or kit disclosed herein can structurally resemble a flavonoid. In some instances, a compound, composition, method, or kit herein may not have effect on plant in absence of microbes.

In some instances, disclosed herein is a drug discovery approach for agriculture, which uses synthetic biology, high throughput screening, and big data analytics to rapidly identify and optimize molecular inputs to close the yield gap. The research areas disclosed herein include photosynthesis, shoot architecture, water capture and efficiency, nutrient uptake, and root architecture. With four seasons of both independent and internal field trials on broadacre crops such as corn, soy, and cereals and specialty crops such as tomatoes and lettuce, the data show that a compound, composition, method, or kit herein are an effective and reliable yield amplifier and produce climate resilient crops.

In some instances, a compound, composition, method, or kit herein can enable broadacre crops such as corn, soy, and wheat to access nutrients previously inaccessible to control plants (without the help of the compound, composition, method, or kit herein). In some instances, the compound, composition, method, or kit disclosed herein not only increase yield performance, but also result in healthier plants and larger, high-quality crops, for example corns. Corn field trials in Buckingham, Iowa have shown that the compound, composition, method, or kit herein lessens the damaging effects of nitrogen deficiency, and help promote healthy plant growth and ear development. The nitrogen content of plants can be quantified with a tissue sample. In a standard tissue sample test of plants with zero nitrogen applies, the compound, composition, method, or kit can lead to higher nitrogen content. The same effect can be seen in drone imagery across large scale strip trials in corn at tasseling. For example, plots treated with a compound, salt, or formulation as described herein can show healthier plants across the treated strip. Lab trials for example with wheat on a nutrient-stressed substrate show that plants treated with the compound, composition, method, or kit herein can access nutrients unavailable to control plants. The plant's ability to thrive when challenged with nutrient stress supports vigorous germination and emergence.

In some instances, incorporation of a compound, composition, method, or kit herein results in a higher proportion of larger produce at harvest, in addition to the yield increase.

### Definitions

Unless defined otherwise, all technical and scientific terms used herein have the same meaning as commonly understood by one of the ordinary skill in the art to which this disclosure belongs. Unless mentioned otherwise, the techniques employed or contemplated herein are standard methodologies. The materials, methods and examples are illustrative only and not limiting.

The details of one or more embodiments are set forth in the accompanying drawings, the claims, and the description herein. Other features, objects, and advantages of the inventive embodiments disclosed and contemplated herein can be combined with any other embodiment unless explicitly excluded.

The open terms for example “contain,” “containing,” “include,” “including,” and the like can mean comprising.

The singular forms “a”, “an”, and “the” as used herein can include plural references unless the context clearly dictates otherwise.

Unless otherwise indicated, some instances herein contemplate numerical ranges. When a numerical range is provided, unless otherwise indicated, the range can include the range endpoints. Unless otherwise indicated, numerical ranges can include all values and subranges therein as if explicitly written out.

The term “about” in relation to a reference numerical value can include a range of values plus or minus 10% from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

The term “compounds” can refer to compounds encompassed by generic formulae disclosed herein, any subgenus of those generic formulae, and any specific compounds within those generic or subgeneric formulae. The compounds can be a specific species, a subgenus or larger genus identified either by their chemical structure and/or chemical name. Further, compounds also include substitutions or modifications of any of such species, subgenuses or genuses, which are set forth herein. When the chemical structure and chemical name conflict, the chemical structure can be determinative of the identity of the compound. The compounds can contain one or more chiral centers and/or double bonds and therefore, can exist as stereoisomers, isomers, enantiomers or diastereomers. Accordingly, the chemical structures within the scope of the specification encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Further, when partial structures of the compounds are illustrated, asterisks indicate the point of attachment of the partial structure to the rest of the molecule. Enantiomeric and stereoisomeric mixtures can be resolved into their component enantiomers or stereoisomers using separation techniques or chiral synthesis techniques well known to the skilled artisan. The compounds can include any salt or solvate forms of the compounds. The compounds can include any derivatives of the compounds.

The term “derivative” can be used interchangeably with the term “analog.” Compound A can be a derivative or analog of compound B if 1, 2, 3, 4, or 5 atoms of compound A is replaced by another atom or a functional group (e.g., amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, or substituted or unsubstituted heterocycloalkyl) to form compound B. The term “derivative” can also refer to a chemical compound that is structurally similar to another but differs slightly in composition (as in the replacement of one atom by an atom of a different element or in the presence of a particular functional group)

The term “isolated” can refer to a form isolated from a mixture, e.g., soil, or a substantially purified form, e.g., a high content of 80% or more w/w of all ingredients other than water, or of all active ingredients.

The term “solvate” can include, but is not limited to, a solvate that retains one or more of the activities and/or properties of the compound and that is not undesirable. Examples of solvates include, but are not limited to, a compound in combination with water, isopropanol, ethanol, methanol, DMSO, ethyl acetate, acetic acid, ethanolamine, or combinations thereof.

The term “salt” can include, but are not limited to, salts that retain one or more of the activities and properties of the free acids and bases and that are not undesirable.

Illustrative examples of salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, y-hydroxybutyrates, glycolates, tartrates, methanesulfonates, propanesulfonates, naphthalene-1-sulfonates, naphthalene-2-sulfonates, and mandelates.

Unless otherwise indicated, a chemical structure can refer to any compound having the chemical structure.

Unless otherwise indicated, formulations herein can be powdery.

Unless otherwise indicated, powder formulations herein can contain water in an amount from about 0% to about 15% w/w, for example 0-10%, 0-5%, or 0-1% w/w; or about: 1%, 2%, 3%, 4%, 5%, 6%, 7%, 8%, 9%, 10, 1%, 12%, 13%, 14%, 15%, 20%, 25%, 30%, 35%, 40%, 45%, 50%, 55%, 60%, 65%, 70%, 75%, 80%, 85%, 90% or 99% w/w, based on the weight of the formulation.

Unless otherwise indicated, whenever there is a stereocenter in a structure disclosed or illustrated herein, the stereocenter can be R or S in each case.

Unless otherwise indicated, whenever there is a symbol

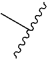

when used as part of a molecular structure herein can refer to a single bond.

The term “amino” can refer to functional groups that contain a basic nitrogen atom with a lone pair. For example, amino can include the radical

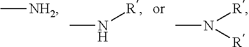

wherein each R′ is independently H, halo, alkyl, aryl, heteroalkyl, arylalkyl, heteroaryl, heteroarylalkyl, cycloalkyl, or heterocycloalkyl.

The term “halo” or “halogen” can refer to fluorine, chlorine, bromine or iodine or a radical thereof.

The term “alkyl” can refer to a saturated or unsaturated, branched, straight-chain or cyclic monovalent hydrocarbon group derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl; and the like.

The term “aryl” can refer to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain instances, an aryl group comprises from 6 to 20 carbon atoms.

The terms “heteroalkyl, heteroalkanyl, heteroalkenyl, heteroalkynyl” refer to alkyl, alkanyl, alkenyl and alkynyl groups, respectively, in which one or more of the carbon atoms (and any associated hydrogen atoms) are each independently replaced with the same or different heteroatomic groups. Typical heteroatomic groups include, but are not limited to, —O—, —S—, —O—O′, —S—S—, —O—S—, —NR′—, ═N—N═, —N═N—, —N═N—NR′—, —PH—, —P(O)₂—, —O—P(O)₂—, —S(O)—, —S(O)₂—, —SnH₂— and the like, wherein R′ is hydrogen, alkyl, substituted alkyl, cycloalkyl, substituted cycloalkyl, aryl or substituted aryl.

The term “heteroaryl” can refer to a monovalent heteroaromatic group derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain instances, the heteroaryl group is from 5-20 membered heteroaryl, and in other instances is from 5-10 membered heteroaryl. In certain instances heteroaryl groups are those derived from thiophene, pyrrole, benzothiophene, benzofuran, indole, pyridine, quinoline, imidazole, oxazole and pyrazine.

The term “arylalkyl” can refer to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In certain instances, an arylalkyl group is (C₆-C₃₀) arylalkyl, e.g., the alkanyl, alkenyl or alkynyl moiety of the arylalkyl group is (C₁-C₁₀) and the aryl moiety is (C₆-C₂₀).

The term “heteroaryl” can refer to a monovalent heteroaromatic group derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain instances, the heteroaryl group is from 5-20 membered heteroaryl, and in other instances is from 5-10 membered heteroaryl. In certain instances heteroaryl groups are those derived from thiophene, pyrrole, benzothiophene, benzofuran, indole, pyridine, quinoline, imidazole, oxazole and pyrazine.

The term “heteroarylalkyl” can refer to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heteroarylalkynyl is used. In certain instances, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl.

The term “cycloalkyl” can refer to a saturated or unsaturated cyclic alkyl group. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. In certain instances, the cycloalkyl group is (C₃-C₁₀) cycloalkyl, or in certain instances (C₃-C₆) cycloalkyl.

The term “heterocycloalkyl” can refer to a saturated or unsaturated cyclic alkyl group in which one or more carbon atoms (and any associated hydrogen atoms) are independently replaced with the same or different heteroatom. Typical heteroatoms to replace the carbon atom(s) include, but are not limited to, N, P, O, S, and Si. Typical heterocycloalkyl groups include, but are not limited to, groups derived from epoxides, imidazolidine, morpholine, piperazine, piperidine, pyrazolidine, pyrrolidine, quinuclidine, and the like.

The term “diastereomeric excess” (DE) can refer to the difference from the relative abundance of two diastereomers. For instance, if there are two diastereomers and their mole or weight percentages are A and B, then DE can be calculated as: DE=[(A−B)/(A+B)]*100%. For example, if a mixture contains 75% of one diastereomer and 25% of the other diastereomer, the diastereomeric excess is 50%. In another example, if a mixture that is 95% of one diastereomer, the diastereomeric excess is 90%.

The term “enantiomeric excess” (EE) can refer to the difference from the relative abundance of two enantiomers. For instance, if there are two enantiomers and their mole or weight percentages are A and B, then EE can be calculated as: EE=[(A−B)/(A+B)]*100%. For example, if a mixture contains 75% of one enantiomer and 25% of the other enantiomer, the enantiomeric excess is 50%. In another example, if a mixture that is 95% of one enantiomer, the enantiomeric excess is 90%.

The term “substituted” can refer to a group in which one or more hydrogen atoms are each independently replaced with the same or different substituent(s). Typical substituents include, but are not limited to halo, alkyl, aryl, heteroalkyl, arylalkyl, heteroaryl, heteroarylalkyl, cycloalkyl, and heterocycloalkyl.

Unless otherwise indicated, “treated” can refer to “contacted.” Similarly, “untreated” can refer to “uncontacted.”

The term “substantially identical plant” can refer to a plant of the same species as an earlier referenced plant. For example, a substantially identical but otherwise uncontacted plant belongs to the same species as a contacted plant. The substantially identical but otherwise uncontacted plant can have a height of about 80% to 120% of the contacted plant (as measured from the surrounding soil to the highest point of the plant) and/or can have a mass of about 80% to 120% of the contacted plant.

The term “drought” can mean conditions with less than 20 inches, 15 inches, 10 inches, or 5 inches of rainfall within the past 12 months. The term “drought” can also mean conditions with a Palmer Drought Severity Index (PDSI) of less than −1.0. The term “adequately irrigated condition” can mean a condition with more than 20 inches of rainfall within the past 12 months. The term “adequately irrigated condition” can mean a condition with a PDSI of more than −1.0.

The term “plant” can be used interchangeably with the term “crop” and can include, but is not limited to any crop, cultivated plant, fungus, or alga that is harvested for food, clothing, livestock fodder, biofuel, medicine, or other uses. For example, plants include field and greenhouse crops, including but not limited to broad acre crops, fruits and vegetables, perennial tree crops, and ornamentals. Plants include, but are not limited to sugarcane, pumpkin, maize (corn), wheat, rice, cassava, soybeans, hay, potatoes, cotton, tomato, alfalfa, and green algae. Plants also include, but are not limited to any vegetable, such as cabbage, turnip, turnip, carrot, parsnip, beetroot, lettuce, beans, broad beans, peas, potato, eggplant, tomato, cucumber, pumpkin, squash, onion, garlic, leek, pepper, spinach, yam, sweet potato, and cassava.

Introduction

Phosphorous and nitrogen are critical or limiting elements for plants in agricultural system. Although agricultural soils are frequently supplemented with phosphorus and nitrogen-rich fertilizers, a large fraction of these vital elements become rapidly unavailable to plants through immobilization, leaching, degradation, or fixation. Excess nutrients from fertilizers pollute water-ways, are dependent on non-renewable resources, and contribute greenhouse gases to the environment. Thus, the future of sustainable agriculture depends upon new technologies that will reduce the amount of fertilizer inputs while maintaining or increasing yield. Soil microbes play a major role in delivering plant-required nutrients, such as phosphorus and nitrogen, from the soil to plants. These microbial-based nutrient transfer processes are under-utilized in modern large scale agriculture, and to date a scalable and effective solution to improving the soil's innate ability to increase orthophosphate and fixed nitrogen in the soil has yet to be developed for broad-acre farming (Lucy 2004).

A major mode of bacterial phosphate solubilization is the secretion of organic acids. This natural process is massively under-utilized in modern large scale agriculture, and to date a dependable and effective solution to improving the soil's innate microbial orthophosphate production has yet to be developed for broad-acre farming. If the bacterial capability to enhance the pool of available orthophosphates in the soil is increased, agricultural systems would experience enhanced plant growth while limiting the application of expensive and inefficient chemical fertilizers. Disclosed herein are compounds and formulation that cause a significant increase in phosphate solubilization of soil microbes, both in soil bacterium in isolated liquid culture and in the soil's innate microbial community.

The efforts to improve bacterial phosphate solubilization and nitrogen fixation in soils has relied upon the introduction of microbial inoculants into the soil. This strategy has been ineffective in broad-acre farming and has several disadvantages: 1.) The viability of live microbes is reduced when bottled and not maintained in proper growth conditions 2.) Many beneficial soil microorganisms cannot be cultured 3.) The persistence and bioactivity of added soil microbes may be low due to being out-competed by the native, established soil microbial population and 4.) There are complex regulatory requirements and restrictions in the introduction of microbes to the environment.

An orthogonal approach to microbial inoculants is leveraging the capacity of the endogenous soil microbiome to enhance plant growth promoting activities. Symbiosis between the root and soil microbiome is initiated and maintained through small molecule signaling. Small molecules that can serve as universal signals to native microbes to increase agronomically important activities is a potential solution for a standardized method to improve the soil's ability to provide nutrients to plants. Plants use chemical signaling within their tissues and organs to communicate and respond to environmental cues, such as light, water, nutrients, beneficial microbes, and pathogenic microbes. There are various categories/classes of chemical signals plants employ. Chemical signals can be moved between roots and shoots through the waterways of the plant, can be excreted into the soil, and in some cases even as gases in the air.

Compounds, salts, solvates, and/or formulations described herein can be applied to a soil or a plant (e.g., to the seed, roots, or canopy of the plant). Compounds, salts, solvates, and/or formulations described herein can result in an increase in available phosphates in the soil, by stimulating the activity of phosphate solubilizing bacteria. Compounds, salts, solvates, and/or formulations described herein can result in an increase in available nitrogen in the soil, by stimulating the activity of nitrogen fixing bacteria. Disclosed herein are the compounds and formulations that can improve available soil phosphate and nitrogen. Also disclosed herein are methods of making the compounds and/or formulations and methods of using the compounds and/or formulations.

Compounds, salts, solvates, and/or formulations described herein can be present with a microbe (e.g., bacteria, actinomycetes, fungi, or protozoa). In some instances, the microbe comprises an isolated bacterium (e.g., purified, or substantially purified). In some instances, the microbe comprises a bacterium from an inoculated or cultured soil. In some instances, the microbe is present in at least about 10 (e.g., at least about 100 or at least about 1000) colony forming units per gram of the agricultural formulation. In some instances, the microbe comprises a wild-type bacterium. In some instances, the microbe comprises a genetically engineered bacterium. In some instances, the microbe comprises a phosphate solubilizing bacterium, a nitrogen fixing bacterium, or a combination thereof. In some instances, the phosphate solubilizing bacterium comprises a bacteria strain of the genus Bacillus. In some instances, the bacteria strain of the genus Bacillus comprises Bacillus megatarium. In some instances, the nitrogen fixing bacterium comprises Azotobacter vinlandii. In some instances, a microbe comprises at least one Gram negative cell. In some instances, the at least one Gram negative cell comprises a Gram negative cocci, a Gram negative bacillus, or a combination thereof. In some instances, the microbe comprises at least one Gram positive cell. In some instances, the at least one Gram positive cell comprises a Gram positive cocci, a Gram positive bacillus, or a combination thereof. In some instances, the microbe comprises at least one member selected from the group consisting of chlamydiae, green nonsulfure bacteria, acinobacteria, planctomycetes, spirochaetes, fusobacteria, cyanobacteria, thermophilic bacteria, acidobacteria, proteobacteria, Azotobacter chroococcum, Pseudomonas stutzeri, Pseudomonas pseudoalcaligenes, Massilia tieshanesis, Massilia aerilata, Massilia putida, Bacillus solisilvae, Bacillus niacini, Massilia agilis, Bacillus wiedmannii, Massilia brevitalea, Bacillus acidiceler, Bacillus toyonensis, Pseudomonas otitidis, Pseudomonas citronellolis, Paenibacillus qinlingensis, Massilia solisilvae, Massilia terrae, Bacillus paramycoides, Massilia aurea, Bacillus acidicola, Panenibacillus alginolyticus, Bacillus novalis, Pseudomonas aeruginosa, Bacillus halmapalus, Pseudomonas knackmussii, Massilia arvi, Massilia agri, Massilia pinisoli, Bacillus megaterium, Bacillus bataviensis, Massilia chloroacetimidivorans, Bacillus mycoides, Bacillus flexus, Bacillus simplex, Pseudomonas balearica, Pseudomonas plecoglossicida, Caballeronia turbans, Psychobacillus lasiicaptis, Bacillus soli, Bacillus cohnii, Cupriavidus campinensis, Brevibacterium frigoritolerans, Bacillus pocheonensis, Pseudomonas monteilii, Bacillus vireti, Bacillus pacificus, Paenibacillus taihuensis, Azotobacter beijerinckii, Paenibacillus contaminans, Bacillus drentensis, Bacillus thuringiensis, Bacillus firmus, Bacillus cereus, Bacillus mobilis, Bacillus luciferensis, Massilia niastensis, Bacillus cucumis, Pseudomonas flavescens, Massilia timonae, Massilia kyonggiensis, Pseudomonas indica, Bacillus phyllosphaerae, Pseudomonas guguanensis, Paenibacillus beijingensis, Bacillus pseudomycoides, Adhaeribacter terreus, Microvirga zambiensis, Pseudomonas oryzae, and any combination thereof.

Compounds

Disclosed herein are compounds or salts thereof of Formula I:

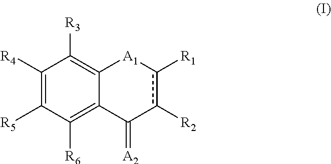

or any salt or solvate thereof,

wherein A₁ and A₂ can independently be O or S, R₁ and R₂ can independently be —H, —OH, —SH, —COOH, C₁-C₆ alkyl, C₃-C₆ cycloalkyl, or —Xₚ, where —Xₚ is:

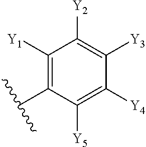

wherein Y₁, Y₂, Y₃, Y₄, and Y₅ can independently be —H, —OH, —SH, —F, —Cl, —Br, —I, or —O—Z₁, wherein Z₁ can be C₁-C₄ alkyl, or

wherein R₁ and R₂ along with the carbon atoms connecting them can form a five or six-membered cycloalkyl ring or cycloalkenyl ring, or a five or six-membered aryl ring; and R₃, R₄, R₅, and R₆ can independently be —H, —OH, —F, —Cl, —Br, —I, or —SH.

Also disclosed herein are compounds or salts thereof of Formula II:

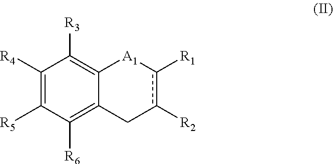

or any salt or solvate thereof,

wherein A₁ and A₂ can independently be O or S, R₁ and R₂ can independently be —H, —OH, —SH, —COOH, C₁-C₆ alkyl, C₃-C₆ cycloalkyl, or —Xₚ, where —Xₚ is:

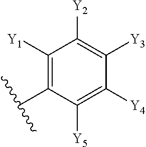

wherein Y₁, Y₂, Y₃, Y₄, and Y₅ can independently be —H, —OH, —SH, —F, —Cl, —Br, —I, or —O—Z₁, wherein Z₁ can be C₁-C₄ alkyl, or

wherein R₁ and R₂ along with the carbon atoms connecting them can form a five or six-membered cycloalkyl ring or cycloalkenyl ring, or a five or six-membered aryl ring; and R₃, R₄, R₅, and R₆ can independently be —H, —OH, —F, —Cl, —Br, —I, or —SH.

Also disclosed herein are compounds or salts thereof of Formula III:

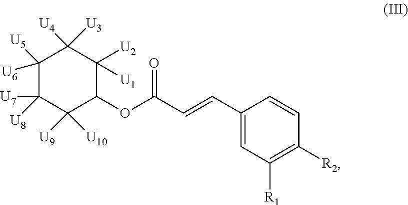

or any salt or solvate thereof, wherein:
   R₁ and R₂ can independently be —H, —OH, —COOH, —SH, C₁-C₆ alkyl, C₃-C₆ cycloalkyl, or —Xₚ, wherein —Xₚ can be:

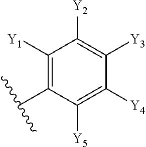

wherein Y₁, Y₂, Y₃, Y₄, and Y₅ can independently be —H, —OH, —SH, —F, —Cl, —Br, —I, or —O—Z₁, wherein Z₁ is C₁-C₄ alkyl, or
wherein R₁ and R₂ along with the carbon atoms connecting them can form a five or six-membered cycloalkyl ring or cycloalkenyl ring, or a five or six-membered aryl ring; and
U₁, U₂, U₃, U₄, U₅, U₆, U₇, U₈, U₉, and U₁₀ can independently be —H, —OH, —COOH, —SH, —F, —Cl, —Br, —I, —COO—Z₁, or —O—Z₁, wherein Z₁ can be C₁-C₄ alkyl.

In some instances, a compound can be of Formula Ia, Formula Ib, Formula Ic, or Formula Id:

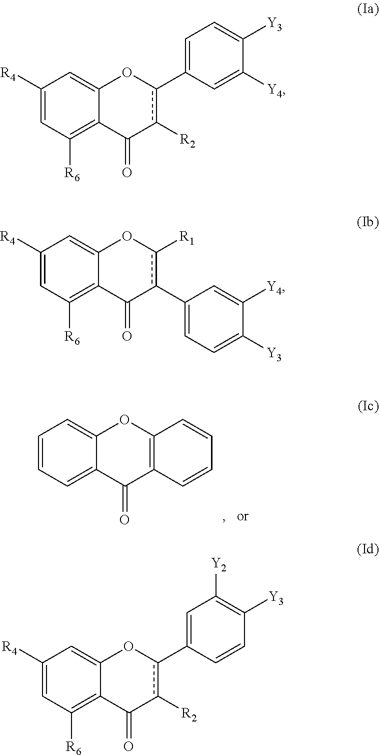

wherein R₁ and R₂ can independently be —H, —OH, —SH, COOH, C₁-C₆ alkyl, or C₃-C₆ cycloalkyl; Y₃ and Y₄ can independently be —H, —OH, —SH, —F, —Cl, —Br, —I, or —O—Z₁, wherein Z₁ can be C₁-C₄ alkyl, and R₄ and R₆ can independently be —H, —OH, —F, —Cl, —Br, —I, or —SH.

In some cases, a compound or salt thereof can be of Formula Ia. In some cases, a compound of Formula Ia can include:

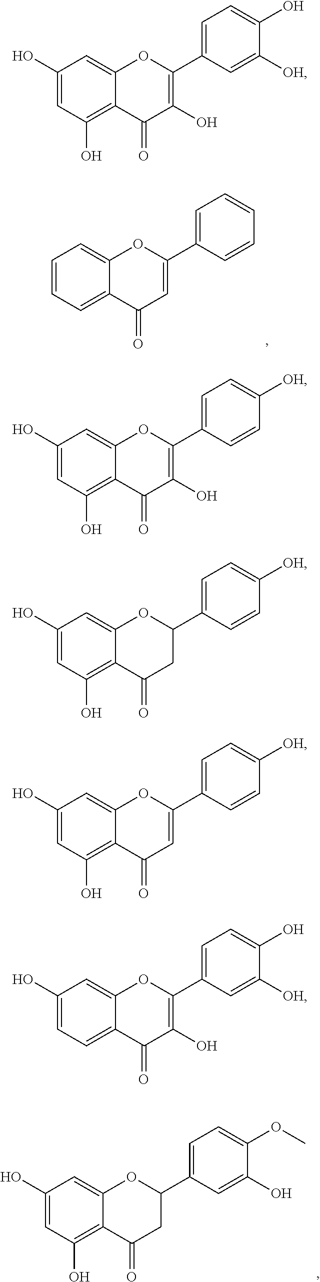

or a salt of any of these.

In some cases, a compound can be of Formula Ib or a salt thereof. In some cases, a compound of Formula Ib can include:

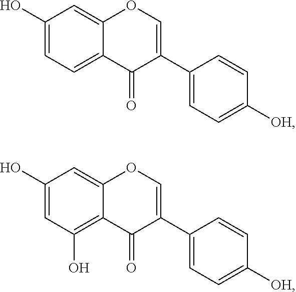 [SMILES: O=c1c(-c2ccc(O)cc2)coc2cc(O)ccc12 ; O=c1c(-c2ccc(O)cc2)coc2cc(O)cc(O)c12]

or a salt of any of these.

In some cases, a compound can be of Formula Ic or a salt thereof:

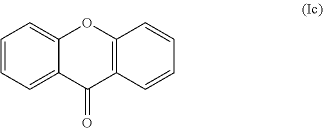 [SMILES: O=c1c2ccccc2oc2ccccc12]

In some cases, a compound can be of Formula Id or a salt thereof. In some cases, a compound of Formula Id can include:

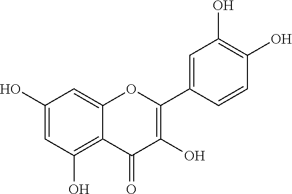 [SMILES: O=c1c(O)c(-c2ccc(O)c(O)c2)oc2cc(O)cc(O)c12]

or a salt of this.

In some cases, a compound or salt thereof can be of Formula IIa:

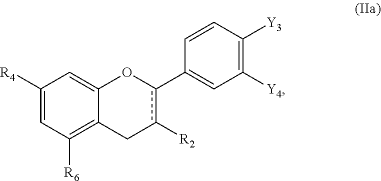

wherein R₂ can independently be —H, —OH, —SH, C₁-C₆ alkyl, or C₃-C₆ cycloalkyl; Y₃ and Y₄ can independently be —H, —OH, —SH, —F, —Cl, —Br, —I, or —O—Z₁, wherein Z₁ can be C₁-C₄ alkyl, and R₄ and R₆ can independently be —H, —OH, —F, —Cl, —Br, —I, or —SH.

In some cases, a compound or salt thereof of Formula IIa can include:

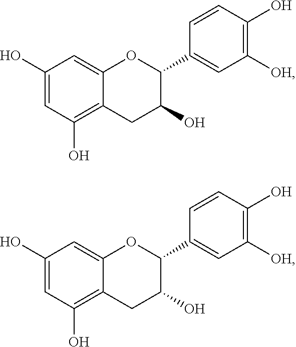 [SMILES: Oc1cc(O)c2c(c1)O[C@H](c1ccc(O)c(O)c1)[C@@H](O)C2 ; Oc1cc(O)c2c(c1)O[C@H](c1ccc(O)c(O)c1)[C@H](O)C2]

or a salt of either of these.

In some cases, a compound or salt thereof can be of Formula IIIa:

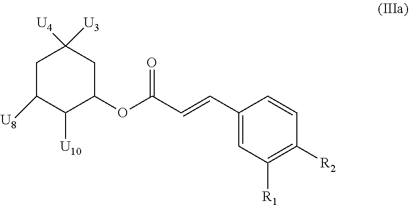

or any salt or solvate thereof, wherein:
   R₁ and R₂ can independently be —H, —OH, —COOH, —SH, C₁-C₆ alkyl, C₃-C₆ cycloalkyl, or —Xₚ, wherein —Xₚ can be:

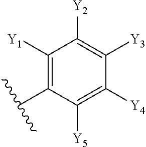

wherein Y₁, Y₂, Y₃, Y₄, and Y₅ can independently be —H, —OH, —SH, —F, —Cl, —Br, —I, or —O—Z₁, wherein Z₁ is C₁-C₄ alkyl, or
wherein R₁ and R₂ along with the carbon atoms connecting them can form a five or six-membered cycloalkyl ring or cycloalkenyl ring, or a five or six-membered aryl ring; and
U₃, U₄, U₈, and U₁₀ can independently be —H, —OH, —COOH, —SH, —F, —C₁, —Br, —I, —COO—Z₁, or —O—Z₁, wherein Z₁ can be C₁-C₄ alkyl.

In some cases, a compound or salt thereof of Formula IIIa can include:

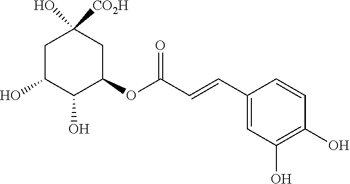 [SMILES: O=C(/C=C/c1ccc(O)c(O)c1)O[C@@H]1C[C@](O)(C(=O)O)C[C@@H](O)[C@H]1O]

or a salt or solvate thereof.

In some instances, a compound, salt, or solvate can include any isomer. In some instances, a compound, salt, or solvate can include any stereoisomer. In some instances, a compound, salt, or solvate can be a tautomer of a compound, salt, or solvate disclosed herein.

In some instances, a compound, salt, or solvate can be a diastereoisomer. In some instances, a compound, salt, or solvate can be a diastereoisomer having a diastereomeric excess of at least about 50%, 60%, 70%, 80%, 85%, 90%, 95%, or from at least about 50% to 100%. A compound, salt, or solvate disclosed herein, may have a diastereomeric excess of at least about 15%, 20%, 30%, 40%, 50%, 60%, 70%, 80%, 85%, 90%, 95%, or 99%. A compound, salt, or solvate disclosed herein, may have a diastereomeric excess of about 15%-99%, 20%-99%, 30%-99%, 40-99%, 50-99%, 60-99%, 70-99%, 80-99%, 90-99%, 15%-90%, 20%-90%, 30%-90%, 40-90%, 50-90%, 60-90%, 70-90%, 80-90%, 15%-80%, 20%-80%, 30%-80%, 40-80%, 50-80%, 60-80%, 70-80%, 15%-70%, 20%-70%, 30%-70%, 40-70%, 50-70%, 60-70%, 15%-60%, 20%-60%, 30%-60%, 40-60%, 50-60%, 15%-50%, 20%-50%, 30%-50%, 40-50%, 15%-40%, 20%-40%, 30%-40%, 15%-30%, 20%-30%, or 15-20%. In some instances, a compound, salt, or solvate disclosed herein, may have a diastereomeric excess of from at least about 50% to 100%.

In some instances, a compound, salt, or solvate can include any enantiomer thereof. In some instances, a compound, salt, or solvate can be an enantiomer having an enantiomeric excess of at least about 50%, 60%, 70%, 80%, 85%, 90%, 95%, or from at least about 50% to 100%. A compound, salt, or solvate disclosed herein, may have an enantiomeric excess of at least about 15%, 20%, 30%, 40%, 50%, 60%, 70%, 80%, 85%, 90%, 95%, or 99%. A compound, salt, or solvate disclosed herein, may have an enantiomeric excess of about 15%-99%, 20%-99%, 30%-99%, 40-99%, 50-99%, 60-99%, 70-99%, 80-99%, 90-99%, 15%-90%, 20%-90%, 30%-90%, 40-90%, 50-90%, 60-90%, 70-90%, 80-90%, 15%-80%, 20%-80%, 30%-80%, 40-80%, 50-80%, 60-80%, 70-80%, 15%-70%, 20%-70%, 30%-70%, 40-70%, 50-70%, 60-70%, 15%-60%, 20%-60%, 30%-60%, 40-60%, 50-60%, 15%-50%, 20%-50%, 30%-50%, 40-50%, 15%-40%, 20%-40%, 30%-40%, 15%-30%, 20%-30%, or 15-20%. In some instances, a compound, salt, or solvate disclosed herein, may have an enantiomeric excess of from at least about 50% to 100%.

Compositions

Also disclosed herein are compositions that can comprise one or more compounds, salts or solvates as described herein. In some cases, a composition can be a solid composition. In some cases, a composition can be a liquid composition. A composition can be used as a seed treatment, soil drench, granule formulation, or foliar spray to improve the productivity of a wide variety of crops.

A composition as described herein containing one or more compounds, salts or solvates described herein can increase an amount of phosphate solubilization in a soil. For example, a composition can comprise an amount of a compound, salt, or solvate that is sufficient to increase an amount of soluble orthophosphate produced from an insoluble phosphate source (such as tricalcium phosphate or equivalent) by one or more live microbes present in soil. A composition as described herein containing one or more compounds, salts or solvates described herein can increase an amount of available nitrogen in a soil. For example, a composition can comprise an amount of a compound, salt, or solvate that is sufficient to increase an amount of nitrogen fixation from one or more live microbes present in soil. A composition as described herein containing one or more compounds, salts or solvates described herein can increase harvest yield of the plant. A composition as described herein containing one or more compounds, salts or solvates described herein can increase a biomass of the plant. A composition as described herein containing one or more compounds, salts or solvates described herein can increase a level of greenness of the plant.

A composition can comprise at least about 0.1% (w/w) of a compound, salt or solvate, for example, at least about 0.1%, at least about 0.2%, at least about 0.3%, at least about 0.4%, at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of a compound, salt or solvate.

A composition can comprise less than about 95% (w/w) of a compound, salt or solvate, for example, less than about 0.1%, less than about 0.2%, less than about 0.3%, less than about 0.4%, less than about 0.5%, less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, or less than about 95% of a compound, salt or solvate.

A composition can comprise about 0.1%-100% (w/w) of an AB compound, salt or solvate, for example, about 0.1%-1%, 0.1%-5%, about 0.1-10%, about 0.1%-20%, about 0.5%-1%, about 0.5%-5%, about 0.5%-10%, about 0.5%-20%, about 1%-5%, about 1%-10%, about 1%-20%, about 5%-10%, about 5%-20%, about 10%-20%, about 10%-30%, about 20%-30%, about 20%-40%, about 30%-40%, about 30%-50%, about 40%-50%, about 40%-60%, about 50%-60%, about 50%-70%, about 60%-70%, about 60%-80%, about 70%-80%, about 70%-90%, about 80%-90%, about 80%-95%, about 90%-95%, about 90%-99%, about 90%-100%, about 95%-99%, or about 99%-100% of the AB compound, salt or solvate.

A composition can comprise at least about 0.1% (w/w) of a compound of Formula I, Formula II, or Formula III as described herein, or any salt or solvate thereof, for example, at least about 0.1%, at least about 0.2%, at least about 0.3%, at least about 0.4%, at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of a compound of Formula I, Formula II, or Formula III, or any salt or solvate thereof.

A composition can comprise less than about 95% (w/w) of a compound of Formula I, Formula II, or Formula III, or any salt or solvate thereof, for example, less than about 0.1%, less than about 0.2%, less than about 0.3%, less than about 0.4%, less than about 0.5%, less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, or less than about 95% of a compound of Formula I, Formula II, or Formula III, or any salt or solvate thereof.

A composition can comprise about 0.1%-100% (w/w) of a compound of Formula I, Formula II, or Formula III, or any salt or solvate thereof, for example, about 0.1%-1%, 0.1%-5%, about 0.1-10%, about 0.1%-20%, about 0.5%-1%, about 0.5%-5%, about 0.5%-10%, about 0.5%-20%, about 1%-5%, about 1%-10%, about 1%-20%, about 5%-10%, about 5%-20%, about 10%-20%, about 10%-30%, about 20%-30%, about 20%-40%, about 30%-40%, about 30%-50%, about 40%-50%, about 40%-60%, about 50%-60%, about 50%-70%, about 60%-70%, about 60%-80%, about 70%-80%, about 70%-90%, about 80%-90%, about 80%-95%, about 90%-95%, about 90%-99%, about 90%-100%, about 95%-99%, or about 99%-100% of a compound of Formula I, Formula II, or Formula III, or any salt or solvate thereof.

A composition can comprise at least about 0.1% (w/w) of a compound of Formula Ia, Formula Ib, Formula Ic, or Formula Id, any salt or solvate thereof, for example, at least about 0.1%, at least about 0.2%, at least about 0.3%, at least about 0.4%, at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of a compound of Formula Ia, Formula Ib, Formula Ic, or Formula Id, or any salt or solvate thereof.

A composition can comprise less than about 95% (w/w) of a compound of Formula Ia, Formula Ib, Formula Ic, or Formula Id, or any salt or solvate thereof, for example, less than about 0.1%, less than about 0.2%, less than about 0.3%, less than about 0.4%, less than about 0.5%, less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, or less than about 95% of a compound of Formula Ia, Formula Ib, Formula Ic, or Formula Id, or any salt or solvate thereof.

A composition can comprise about 0.1%-100% (w/w) of a compound of Formula Ia, Formula Ib, Formula Ic, or Formula Id, or any salt or solvate thereof, for example, about 0.1%-1%, 0.1%-5%, about 0.1-10%, about 0.1%-20%, about 0.5%-1%, about 0.5%-5%, about 0.5%-10%, about 0.5%-20%, about 1%-5%, about 1%-10%, about 1%-20%, about 5%-10%, about 5%-20%, about 10%-20%, about 10%-30%, about 20%-30%, about 20%-40%, about 30%-40%, about 30%-50%, about 40%-50%, about 40%-60%, about 50%-60%, about 50%-70%, about 60%-70%, about 60%-80%, about 70%-80%, about 70%-90%, about 80%-90%, about 80%-95%, about 90%-95%, about 90%-99%, about 90%-100%, about 95%-99%, or about 99%-100% of a compound of Formula Ia, Formula Ib, Formula Ic, or Formula Id, or any salt or solvate thereof.

A composition can comprise at least about 0.1% (w/w) of a compound of Formula IIa, or any salt or solvate thereof, for example, at least about 0.1%, at least about 0.2%, at least about 0.3%, at least about 0.4%, at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of a compound of Formula IIa, or any salt or solvate thereof.

A composition can comprise less than about 95% (w/w) of a compound of Formula IIa, or any salt or solvate thereof, for example, less than about 0.1%, less than about 0.2%, less than about 0.3%, less than about 0.4%, less than about 0.5%, less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, or less than about 95% of a compound of Formula IIa, or any salt or solvate thereof.

A composition can comprise about 0.1%-100% (w/w) of a compound of Formula IIa, or any salt or solvate thereof, for example, about 0.1%-1%, 0.1%-5%, about 0.1-10%, about 0.1%-20%, about 0.5%-1%, about 0.5%-5%, about 0.5%-10%, about 0.5%-20%, about 1%-5%, about 1%-10%, about 1%-20%, about 5%-10%, about 5%-20%, about 10%-20%, about 10%-30%, about 20%-30%, about 20%-40%, about 30%-40%, about 30%-50%, about 40%-50%, about 40%-60%, about 50%-60%, about 50%-70%, about 60%-70%, about 60%-80%, about 70%-80%, about 70%-90%, about 80%-90%, about 80%-95%, about 90%-95%, about 90%-99%, about 90%-100%, about 95%-99%, or about 99%-100% of a compound of Formula IIa, or any salt or solvate thereof.

A composition can comprise at least about 0.1% (w/w) of a compound of Formula:

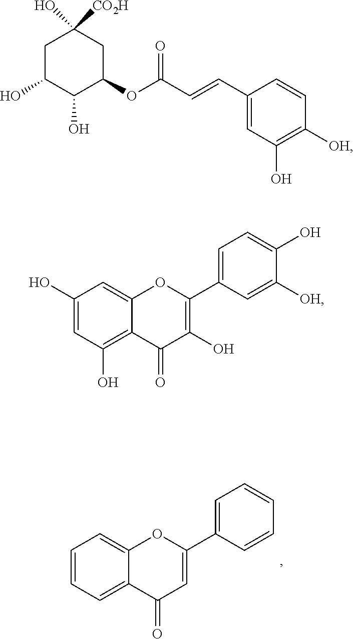 [SMILES: O=C(/C=C/c1ccc(O)c(O)c1)O[C@@H]1C[C@](O)(C(=O)O)C[C@@H](O)[C@H]1O ; O=c1c(O)c(-c2ccc(O)c(O)c2)oc2cc(O)cc(O)c12 ; O=c1cc(-c2ccccc2)oc2ccccc12]

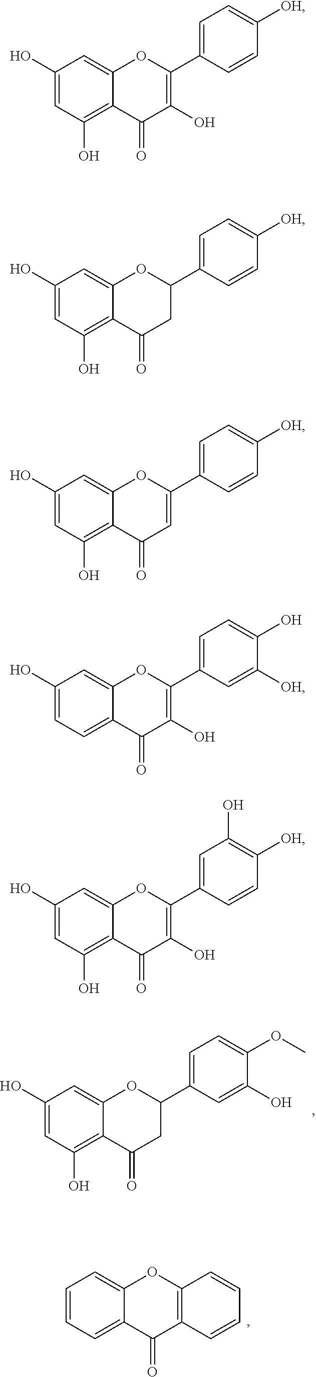

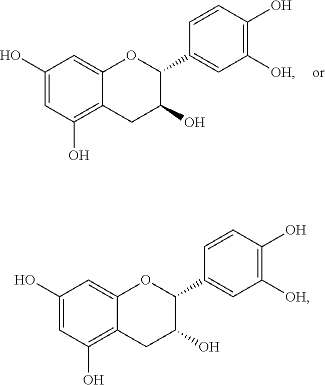 [SMILES: Oc1cc(O)c2c(c1)O[C@H](c1ccc(O)c(O)c1)[C@@H](O)C2 ; Oc1cc(O)c2c(c1)O[C@H](c1ccc(O)c(O)c1)[C@H](O)C2]

or any salt or solvate thereof, for example, at least about 0.1%, at least about 0.2%, at least about 0.3%, at least about 0.4%, at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the compound, or any salt or solvate thereof.

A composition can comprise less than about 95% (w/w) of a compound of Formula:

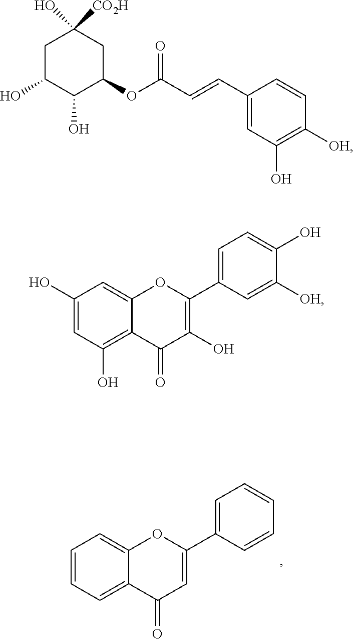 [SMILES: O=C(/C=C/c1ccc(O)c(O)c1)O[C@@H]1C[C@](O)(C(=O)O)C[C@@H](O)[C@H]1O ; O=c1c(O)c(-c2ccc(O)c(O)c2)oc2cc(O)cc(O)c12 ; O=c1cc(-c2ccccc2)oc2ccccc12]

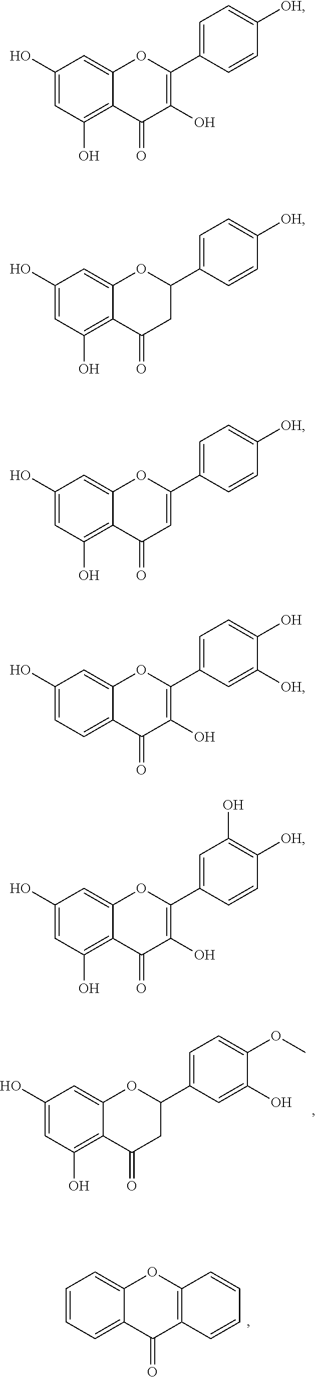

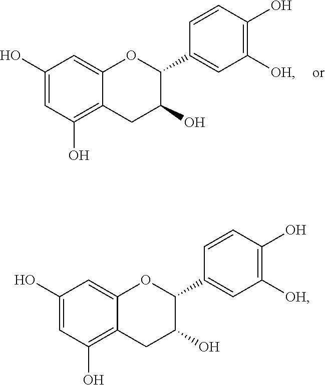 [SMILES: Oc1cc(O)c2c(c1)O[C@H](c1ccc(O)c(O)c1)[C@@H](O)C2 ; Oc1cc(O)c2c(c1)O[C@H](c1ccc(O)c(O)c1)[C@H](O)C2]

or any salt or solvate thereof or any salt or solvate thereof, for example, less than about 0.1%, less than about 0.2%, less than about 0.3%, less than about 0.4%, less than about 0.5%, less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, or less than about 95% of the compound, or any salt or solvate thereof.

A composition can comprise about 0.1%-100% (w/w) of a compound of Formula:

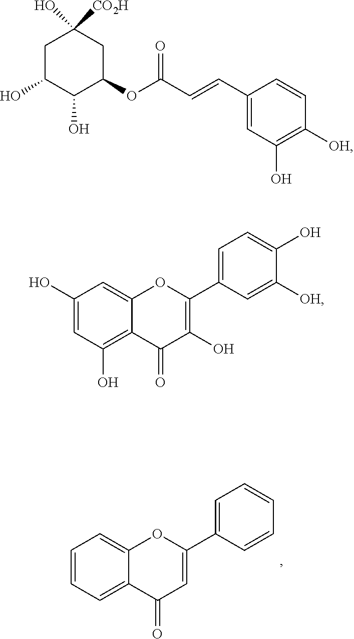 [SMILES: O=C(/C=C/c1ccc(O)c(O)c1)O[C@@H]1C[C@](O)(C(=O)O)C[C@@H](O)[C@H]1O ; O=c1c(O)c(-c2ccc(O)c(O)c2)oc2cc(O)cc(O)c12 ; O=c1cc(-c2ccccc2)oc2ccccc12]

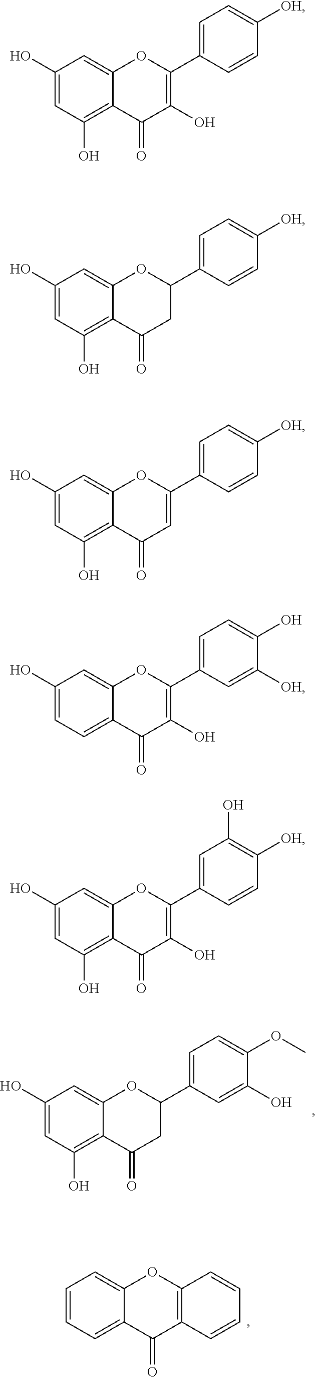

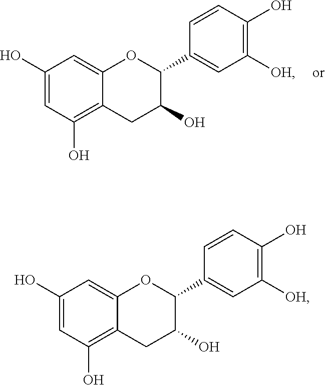 [SMILES: Oc1cc(O)c2c(c1)O[C@H](c1ccc(O)c(O)c1)[C@@H](O)C2 ; Oc1cc(O)c2c(c1)O[C@H](c1ccc(O)c(O)c1)[C@H](O)C2]

or any salt or solvate thereof, or any salt or solvate thereof, for example, about 0.1%-1%, 0.1%-5%, about 0.1-10%, about 0.1%-20%, about 0.5%-1%, about 0.5%-5%, about 0.5%-10%, about 0.5%-20%, about 1%-5%, about 1%-10%, about 1%-20%, about 5%-10%, about 5%-20%, about 10%-20%, about 10%-30%, about 20%-30%, about 20%-40%, about 30%-40%, about 30%-50%, about 40%-50%, about 40%-60%, about 50%-60%, about 50%-70%, about 60%-70%, about 60%-80%, about 70%-80%, about 70%-90%, about 80%-90%, about 80%-95%, about 90%-95%, about 90%-99%, about 90%-100%, about 95%-99%, or about 99%-100% of the compound, or any salt or solvate thereof.

Phosphate Solubilizing Bacteria

Phosphate solubilizing bacteria (PSB) can refer to beneficial bacteria capable of solubilizing inorganic phosphorus from insoluble compounds. Numerous genera and species of phosphate solubilizing bacteria have been described. See, e.g., Y. P. Chen; P. D. Rekha; A. B. Arun; F. T. Shen; W.-A. Lai; C. C. Young (2006). “Phosphate solubilizing bacteria from subtropical soil and their tricalcium phosphate solubilizing abilities”. Applied Soil Ecology. 34 (1): 33-41. In some instances, phosphate solubilizing bacteria refers to a member of an endogenous soil consortium. In some instances, phosphate solubilizing bacteria refers to a non-native phosphate solubilizing bacteria. In some instances, the non-native phosphate solubilizing bacteria is recombinant. In some instances, the non-native phosphate solubilizing bacteria has increased phosphate solubilizing activity relative to a non-recombinant phosphate solubilizing bacteria.

In some instances, agricultural formulations or compositions can comprise from about 10³-10¹¹ cfu of the phosphate solubilizing bacteria per gram of the agricultural formulation. In some instances, agricultural formulations can comprise from about 10⁴-10¹¹cfu of the phosphate solubilizing bacteria per gram of the agricultural formulation. In some instances, agricultural formulations can comprise from about 10⁵-10¹¹ cfu of the phosphate solubilizing bacteria per gram of the agricultural formulation. In some instances, agricultural formulations can comprise from about 10⁶-10¹¹ cfu of the phosphate solubilizing bacteria per gram of the agricultural formulation. In some instances, agricultural formulations can comprise from about 10⁷-10¹¹ cfu of the phosphate solubilizing bacteria per gram of the agricultural formulation. In some instances, agricultural formulations can comprise from about 10⁸-10¹¹ cfu of the phosphate solubilizing bacteria per gram of the agricultural formulation. In some instances, agricultural formulations can comprise from about 10⁹-10¹¹ cfu of the phosphate solubilizing bacteria per gram of the agricultural formulation. In some instances, agricultural formulations can comprise from about 10¹⁰-10¹¹ cfu of the phosphate solubilizing bacteria per gram of the agricultural formulation. In some instances, agricultural formulations can comprise from about 10⁶-10¹⁰ cfu of the phosphate solubilizing bacteria per gram of the agricultural formulation. In some instances, agricultural formulations can comprise from about 10⁶-10⁹ cfu of the phosphate solubilizing bacteria per gram of the agricultural formulation. In some instances, agricultural formulations can comprise from about 10⁶-10⁸ cfu of the phosphate solubilizing bacteria per gram of the agricultural formulation. In some instances, agricultural formulations can comprise from about 10⁶-10⁷ cfu of the phosphate solubilizing bacteria per gram of the agricultural formulation.

A mechanism of mineral phosphate solubilization by PSB strains may involve the release of low molecular weight organic acids, through which their hydroxyl and carboxyl groups chelate the cations bound to phosphate, thereby converting it into soluble forms.

In some instances, phosphate solubilizing bacteria may selected from the genus Bacillus. In some instances, a phosphate solubilizing bacteria may a strain selected from the species Bacillus megaterium.

Nitrogen Fixing Bacteria

Nitrogen-fixing bacteria can refer to bacteria that can covert atmospheric nitrogen to ammonia or other molecules that are available to other living organisms. Nitrogen fixing bacteria can infect root hairs of leguminous plants, such as soybean, clover, alfalfa, string beans and peas. The infection leads to nodule formation within which free nitrogen is converted to combined nitrogen (nitrogen-fixation). Nitrogen fixing bacteria are widespread within domain Bacteria including cyanobacteria (e.g. the highly significant Trichodesmium and Cyanothece), as well as green sulfur bacteria, Azotobacteraceae, rhizobia and Frankia. In some instances, nitrogen fixing bacteria refers to a member of an endogenous soil consortium. In some instances, nitrogen fixing bacteria refers to a non-native nitrogen fixing bacteria. In some instances, the non-native nitrogen fixing bacteria is recombinant. In some instances, the non-native nitrogen fixing bacteria has increased nitrogen fixing activity relative to a non-recombinant nitrogen fixing bacteria.

In some instances, agricultural formulations or compositions can comprise from about 10³-10¹¹ colony forming units (cfu) of the nitrogen fixing bacteria per gram of the agricultural formulation. In some instances, agricultural formulations can comprise from about 10⁴-10¹¹ cfu of the nitrogen fixing bacteria per gram of the agricultural formulation.

In some instances, agricultural formulations can comprise from about 10⁵-10¹¹ cfu of the nitrogen fixing bacteria per gram of the agricultural formulation. In some instances, agricultural formulations can comprise from about 10⁶-10¹¹ cfu of the nitrogen fixing bacteria per gram of the agricultural formulation. In some instances, agricultural formulations can comprise from about 10⁷-10¹¹ cfu of the nitrogen fixing bacteria per gram of the agricultural formulation. In some instances, agricultural formulations can comprise from about 10⁸-10¹¹ cfu of the nitrogen fixing bacteria per gram of the agricultural formulation. In some instances, agricultural formulations can comprise from about 10⁹-10¹¹ cfu of the nitrogen fixing bacteria per gram of the agricultural formulation.

In some instances, agricultural formulations can comprise from about 10¹⁰-10¹¹ cfu of the nitrogen fixing bacteria per gram of the agricultural formulation. In some instances, agricultural formulations can comprise from about 10⁶-10¹⁰ cfu of the nitrogen fixing bacteria per gram of the agricultural formulation. In some instances, agricultural formulations can comprise from about 10⁶-10⁹ cfu of the nitrogen fixing bacteria per gram of the agricultural formulation. In some instances, agricultural formulations can comprise from about 10⁶-10⁸ cfu of the nitrogen fixing bacteria per gram of the agricultural formulation. In some instances, agricultural formulations can comprise from about 10⁶-10⁷ cfu of the nitrogen fixing bacteria per gram of the agricultural formulation.

Excipients, Diluents, and Carriers

A composition disclosed herein can further comprise one or more excipients, diluents, or carriers. An excipient, diluent, or carrier can be one or more pesticides, one or more stabilizers, one or more additives, one or more carriers, one or more dispersants, one or more fertilizer, or any combination thereof. In one example, one or more excipients comprise acetone.

A composition disclosed herein can further comprise one or more pesticides. The pesticide may be a biopesticide. A biopesticide may be a form of a pesticide that can be based on microorganisms or natural products. A biopesticide may include naturally occurring substances that control pests (biochemical pesticides), microorganisms that control pests (microbial pesticides), and pesticidal substances produced by plants containing added genetic material (plant-incorporated protectants) or PIPs. Examples of biopesticides can include, but are not limited to, gluocosinolate, chitosan, spinosad, alkaloids, terpenoids, phenolics, pyrethroids, rotenoids, nicotinoids, strychnine, scilliroside, canola oil and baking soda. The pesticide may be an organophosphate pesticide, carbamate pesticide, organochlorine insecticide, pyrethroid pesticide, sulfonylurea pesticides, or a combination thereof. The pesticide may be a herbicide, algicide, avidicide, bactericide, fungicide, insecticide, miticide, molluscicide, nematicide, rodenticide, virucide, or a combination thereof.

A composition can further comprise one or more stabilizers, polymers, or other additives. The stabilizers, polymers, or additives can include, but are not limited to, penetration agents, adhesives, anticaking agents, dyes, dispersants, wetting agents, emulsifying agents, defoamers, antimicrobials, antifreeze, pigments, colorants, buffers, and carriers. A composition can further comprise surfactants and/or adjuvants.

A composition can comprise one or more diluents. A diluent can be an agriculturally acceptable diluent. In some cases, an agriculturally acceptable diluent can refer to a diluent that, when contacted with a plant in a conventional amount, does not inhibit growth of a plant or cause plant death. In some cases, a diluent can be a plant oil. A plant oil can include sunflower oil, canola oil, avocado seed oil, grapeseed oil, almond oil, cocoa butter, coconut oil, corn oil, cottonseed oil, flax seed oil, hemp oil, olive oil, palm kernel oil, peanut oil, pumpkin seed oil, rice bran oil, safflower oil, sesame seed oil, soybean oil, walnut oil, and any combination thereof.

A composition can comprise one or more carriers. Examples of carriers include, but are not limited to, solid carriers, sponges, textiles, and synthetic materials. The synthetic material may be a porous synthetic material. Additional carriers can include organic carriers, such as waxes, linolin, paraffin, dextrose granules, sucrose granules and maltose-dextrose granules. Alternatively, the carrier can be an anorganic carrier such as natural clays, kaolin, pyrophyllite, bentonite, alumina, montmorillonite, kieselguhr, chalk, diatomaceous earths, calcium phosphates, calcium and magnesium carbonates, sulphur, lime, flours or talc. A composition can be adsorbed into the carrier. The carrier may be characterized by enabling release of the compound, salt, solvate, or formulation.

A composition can further comprise one or more dispersants. The dispersant may be a negatively charged anion dispersant. The dispersant may be a nonionic dispersant.

A composition can further comprise a fertilizer. The fertilizer may be a chemical fertilizer. The fertilizer may be an organic fertilizer. The fertilizer may be an inorganic fertilizer. The fertilizer may be a granulated or powdered fertilizer. The fertilizer may be a liquid fertilizer. The fertilizer may be a slow-release fertilizer.

A composition disclosed herein can be formulated as a dry sprayable formulation. Examples of dry sprayable formulations can include, but are not limited to, wettable powders and water dispersible granules. Wettable powders may comprise compounds, salts, solvates, that have been microionized to powder form. Wettable powders may be applied as suspended particles after dispertion into water. Water dispersible granules may consist of granules that are applied after disintegration or dispersion in water. The water dispersible granules may comprise particles within the range of 0.2 to 4 mm. Water dispersible granules may be formed by agglomeration, spray drying, or extrusion techniques.

A composition can be formulated as a liquid sprayable formulation. Examples of liquid sprayable formulations can include, but are not limited to, soluble concentrates, suspension concentrates, emulsifiable concentrates, microemulsions, oil dispersions, and microencapsulated particles. Suspension concentrates may comprise a stable suspension of the compound, salt, solvate, or formulation in a fluid usually intended for dilution with water before use. Emulsifiable concentrates may comprise a compound, salt, solvate, or formulation with an emulsifying agent in a water insoluble organic solvate which will form an emulsion when added to water. Microemulsions may comprise a compound, salt, solvate, or formulation with an emulsifying agent in a water insoluble organic solvate which will form a solution/emulsion when added to water. In some instances, a liquid formulation herein may comprise an antioxidant, a surfactant or an emulsifier (e.g., ethoxylate, ethoxylated ester, ethoxylated sorbitol ester, polyol alkoxylated ester, a sorbitol-based surfactant, or an alcohol ethoxylate), an oil, water, a lubricant (e.g., polyalkylene glycol), an antifreeze, an antifoam emulsion, a preservative, a thickening agent, or any combination thereof.

A composition can be formulated as a dry spreadable granule formulation. The dry spreadable granule formulation may comprise soil applied granule on inert or fertilizer carriers.

A composition can be formulated as a seed treatment or seed dressing.

A composition can be formulated for rapid release. A composition can be formulated for slow release.

Kits

Also disclosed herein are kits that can comprise a compound, salt, solvate, or composition described herein in a container. In some cases, a kit can further comprise instructions for use. Such instructions can include instructions to perform any step of a method described herein. For example, instructions can include application of a compound, slat, solvate, or composition to a plant, portion thereof, seed thereof, or soil.

A container can include any suitable container for storing a compound, salt, solvate, or composition described herein. A container can also include any suitable container for dispensing a compound, salt, solvate, or composition as described herein. A container can include a spray bottle, a syringe, an ampoule, a vial, a tube, a bucket, a bag, a pouch, or the like.

In some cases, a kit can further comprise other components used in agriculture. For instance, a kit can include soil, fertilizer, pesticide, plant seeds, herbicides, or a live microbe as described herein. In some cases, a kit can comprise any microbe as described herein. In some cases, a microbe can be live microbe. A live microbe in a kit described herein can be a beneficial microbe, a nitrogen fixing microbe, a phosphate solubilizing microbe, or any combination thereof. In some cases, a kit can comprise a spore or inactive microbe. In some cases, a kit can comprise vegetative microbes.

Methods of Increasing Nutrient Availability

Also disclosed herein are methods of increasing soil nutrient availability and/or increasing yield of a plant (e.g. increasing a biomass of a plant, or increasing a greenness of a plant). The methods can comprise contacting a soil or a plant with the compounds, salts, solvates, or compositions disclosed herein.

In some instances, compounds, salts, solvates, or compositions as disclosed herein can directly stimulate the phosphate solubilizing activity of the soil's native microbial consortium (including bacteria strains, actinomycete, fungi, protozoa, and any combination thereof), providing more phosphorus for plant growth. In some instances, phosphate solubilizing microbe disclosed herein can convert insoluble, plant-inaccessible phosphate to soluble, plant-available phosphate. Nitrogen-fixing bacteria (legumes) can convert atmospheric nitrogen to plant available forms of nitrogen.

In some instances, compounds, salts, solvates, and compositions disclosed herein can induce nitrogen fixation of the soil's native microbial consortium (including bacteria strains, actinomycete, fungi, protozoa, and any combination thereof). By thus activating nitrogen fixation in the soil, the compounds, salts, solvates, and compositions disclosed herein can provide extra plant available nutrition. In some instances, compounds, salts, solvates, and compositions disclosed herein can significantly boost crop health and yield in nitrogen limiting environments.

A soil's native microbial consortia can include any number of bacteria strains, actinomycete, fungi, protozoa, or combinations thereof. In some cases, a microbial consortium can comprise live microbes. In some cases, a microbial consortium can comprise dead microbes. In some cases, a microbial consortia can include: Azotobacter chroococcum, Pseudomonas stutzeri, Pseudomonas pseudoalcaligenes, Massilia tieshanesis, Massilia aerilata, Massilia putida, Bacillus solisilvae, Bacillus niacini, Massilia agilis, Bacillus wiedmannii, Massilia brevitalea, Bacillus acidiceler, Bacillus toyonensis, Pseudomonas otitidis, Pseudomonas citronellolis, Paenibacillus qinlingensis, Massilia solisilvae, Massilia terrae, Bacillus paramycoides, Massilia aurea, Bacillus acidicola, Panenibacillus alginolyticus, Bacillus novalis, Pseudomonas aeruginosa, Bacillus halmapalus, Pseudomonas knackmussii, Klebsiella pneumoniae, Klebsiella variicola, Klebsiella oxytoca, Pseudomonas aeruginosa, Serratia marcescens, Bacillus amyloliquefaciens, Gluconacetobacter diazotrophicus Massilia arvi, Massilia agri, Massilia pinisoli, Bacillus megaterium, Bacillus bataviensis, Massilia chloroacetimidivorans, Bacillus mycoides, Bacillus flexus, Bacillus simplex, Pseudomonas balearica, Pseudomonas plecoglossicida, Caballeronia turbans, Psychobacillus lasiicaptis, Bacillus soli, Bacillus cohnii, Cupriavidus campinensis, Brevibacterium frigoritolerans, Bacillus pocheonensis, Pseudomonas monteilii, Bacillus vireti, Bacillus pacificus, Paenibacillus taihuensis, Azotobacter beijerinckii, Paenibacillus contaminans, Bacillus drentensis, Bacillus thuringiensis, Bacillus firmus, Bacillus cereus, Bacillus mobilis, Bacillus luciferensis, Massilia niastensis, Bacillus cucumis, Pseudomonas flavescens, Massilia timonae, Massilia kyonggiensis, Pseudomonas indica, Bacillus phyllosphaerae, Pseudomonas guguanensis, Paenibacillus beijingensis, Bacillus pseudomycoides, Adhaeribacter terreus, Microvirga zambiensis, Pseudomonas oryzae, or any combination thereof.

The compounds, salts, solvates, and compositions disclosed herein can be used in agriculture. The compounds, salts, solvates, and compositions can be used to promote plant growth. The compounds, salts, solvates, and compositions disclosed herein can be used for enhancing shoot stability in plants. The compounds, salts, solvates, and compositions can be used for increasing transport capacity in plants. The compounds, salts, solvates, and compositions can be used for increasing drought tolerance of a plant.

Further disclosed herein are methods of improving agriculture comprising applying a composition (e.g. a liquid composition) comprising a compound, salt, or solvate to a plant (e.g. to a leaf, a root, a stem, or other part of a plant) or a seed thereof, thereby improving agriculture. Improving agriculture can comprise promoting plant growth. Improving agriculture can comprise enhancing shoot stability in plants. Improving agriculture can comprise increasing transport capacity in plants. Improving agriculture can comprise increasing drought tolerance. Improving agriculture can comprise reducing an application of one or more pesticides. Improving agriculture can comprise terminating application of one or more pesticides. Improving agriculture can comprise reducing watering amounts applied to the plants. Improving agriculture can comprise reducing watering frequency to the plants. Improving agriculture can comprise controlling phytopathogenic fungi. Improving agriculture can comprise controlling unwanted plant growth. Improving agriculture can comprise controlling unwanted insect or mite infestation. Improving agriculture can comprise regulating growth of the plant. Improving agriculture can comprise promoting or stimulating activity in one or more fungi.

Compounds, salts, solvates, or compositions described herein can increase plant growth by at least about 5%. The compounds, salts, solvates, or compositions can increase plant growth by at least about 10%. The compounds, salts, solvates, or compositions can increase plant growth by at least about 15%. The compounds, salts, solvates, or compositions can increase plant growth by at least about 20%. The compounds, salts, solvates, or compositions can increase plant growth by at least about 25%. The compounds, salts, solvates, or compositions can increase plant growth by at least about 30%. The compounds, salts, solvates, or compositions can increase plant growth by at least about 50%. The compounds, salts, solvates, or compositions can increase plant growth by at least about 60%, 70%, 80%, 90%, 95%, 100% or more.

The compounds, salts, solvates, or compositions can increase plant growth by at least about 1.5, 2, 3, 4, 5, 6, 7, 8, 9, 10, 11, 12, 13, 14, 15, 16, 17, 18, 19, 20, 25, 30, 40, 50-fold or more. The compounds, salts, solvates, or compositions can increase plant growth by at least about 1.5-fold or more. The compounds, salts, solvates, or compositions can increase plant growth by at least about 2-fold or more. The compounds, salts, solvates, or compositions can increase plant growth by at least about 3-fold or more. The compounds, salts, solvates, or compositions can increase plant growth by at least about 5-fold or more. The compounds, salts, solvates, or compositions can increase plant growth by at least about 10-fold or more. Plant growth or compositions can comprise secondary plant growth.

The compounds, salts, solvates, or compositions can enhance shoot growth by at least about 5%. The compounds, salts, solvates, or compositions can enhance shoot growth by at least about 10%. The compounds, salts, solvates, or compositions can enhance shoot growth by at least about 15%. The compounds, salts, solvates, or compositions can enhance shoot growth by at least about 20%. The compounds, salts, solvates, or compositions can enhance shoot growth by at least about 25%. The compounds, salts, solvates, or compositions can enhance shoot growth by at least about 30%. The compounds, salts, solvates, or compositions can enhance shoot growth by at least about 50%. The compounds, salts, solvates, or compositions can enhance shoot growth by at least about 60%, 70%, 80%, 90%, 95%, 100% or more. The compounds, salts, solvates, or compositions can enhance shoot growth by at least about 1.5, 2, 3, 4, 5, 6, 7, 8, 9, 10, 11, 12, 13, 14, 15, 16, 17, 18, 19, 20, 25, 30, 40, 50-fold or more.

The compounds, salts, solvates, or compositions can enhance shoot growth by at least about 1.5-fold or more. The compounds, salts, solvates, or compositions can enhance shoot growth by at least about 2-fold or more. The compounds, salts, solvates, or compositions can enhance shoot growth by at least about 3-fold or more. The compounds, salts, solvates, or compositions can enhance shoot growth by at least about 5-fold or more. The compounds, salts, solvates, or compositions can enhance shoot growth by at least about 10-fold or more.

The compounds, salts, solvates, or compositions can increase transport capacity in plants by at least about 5%. The compounds, salts, solvates, or compositions can increase transport capacity in plants by at least about 10%. The compounds, salts, solvates, or compositions can increase transport capacity in plants by at least about 15%. The compounds, salts, solvates, or compositions can increase transport capacity in plants by at least about 20%. The compounds, salts, solvates, or compositions can increase transport capacity in plants by at least about 25%. The compounds, salts, solvates, or compositions can increase transport capacity in plants by at least about 30%. The compounds, salts, solvates, or compositions can increase transport capacity in plants by at least about 50%. The compounds, salts, solvates, or compositions can increase transport capacity in plants by at least about 60%, 70%, 80%, 90%, 95%, 100% or more.

The compounds, salts, solvates, or compositions can increase transport capacity in plants by at least about 1.5, 2, 3, 4, 5, 6, 7, 8, 9, 10, 11, 12, 13, 14, 15, 16, 17, 18, 19, 20, 25, 30, 40, 50-fold or more. The compounds, salts, solvates, or compositions can increase transport capacity in plants by at least about 1.5-fold or more. The compounds, salts, solvates, or compositions can increase transport capacity in plants by at least about 2-fold or more. The compounds, salts, solvates, or compositions can increase transport capacity in plants by at least about 3-fold or more. The compounds, salts, solvates, or compositions can increase transport capacity in plants by at least about 5-fold or more. The compounds, salts, solvates, or compositions can increase transport capacity in plants by at least about 10-fold or more.

The compounds, salts, solvates, or compositions can increase drought tolerance in plants by at least about 5%. The compounds, salts, solvates, or compositions can increase drought tolerance in plants by at least about 10%. The compounds, salts, solvates, or compositions can increase drought tolerance in plants by at least about 15%. The compounds, salts, solvates, or compositions can increase drought tolerance in plants by at least about 20%. The compounds, salts, solvates, or compositions can increase drought tolerance in plants by at least about 25%. The compounds, salts, solvates, or compositions can increase drought tolerance in plants by at least about 30%. The compounds, salts, solvates, or compositions can increase drought tolerance in plants by at least about 50%. The compounds, salts, solvates, or compositions can increase drought tolerance in plants by at least about 60%, 70%, 80%, 90%, 95%, 100% or more.

The compounds, salts, solvates, or compositions can increase drought tolerance in plants by at least about 1.5, 2, 3, 4, 5, 6, 7, 8, 9, 10, 11, 12, 13, 14, 15, 16, 17, 18, 19, 20, 25, 30, 40, 50-fold or more. The compounds, salts, solvates, or compositions can increase drought tolerance in plants by at least about 1.5-fold or more. The compounds, salts, solvates, or compositions can increase drought tolerance in plants by at least about 2-fold or more. The compounds, salts, solvates, or compositions can increase drought tolerance in plants by at least about 3-fold or more. The compounds, salts, solvates, or compositions can increase drought tolerance in plants by at least about 5-fold or more. The compounds, salts, solvates, or compositions can increase drought tolerance in plants by at least about 10-fold or more.

The compounds, salts, solvates, or compositions can reduce the application of one or more pesticides. Reducing the application of one or more pesticides can comprise reducing an amount of the one or more pesticides that are applied to the plant. The amount of the one or more pesticides applied to the plant can be reduced by at least about 1%, 5%, 10%, 15%, 20%, 25%, 30%, 35%, 40%, 50%, 60%, 70%, 80%, 90%, 95%, or 100%. The amount of the one or more pesticides applied to the plant can be reduced by at least about 10%. The amount of the one or more pesticides applied to the plant can be reduced by at least about 20%. The amount of the one or more pesticides applied to the plant can be reduced by at least about 30%. The amount of the one or more pesticides applied to the plant can be reduced by at least about 50%.

Alternatively, or additionally, reducing the application of the one or more pesticides can comprise reducing a frequency of which the one or more pesticides are applied to the plant. The frequency of which the one or more pesticides are applied to the plant can be reduced by at least about 1%, 5%10%, 15%, 20%, 25%, 30%, 35%, 40%, 50%, 60%, 70%, 80%, 90%, 95%, or 100%. The frequency of which the one or more pesticides are applied to the plant can be reduced by at least about 10%. The frequency of which the one or more pesticides are applied to the plant can be reduced by at least about 20%. The frequency of which the one or more pesticides are applied to the plant can be reduced by at least about 30%. The frequency of which the one or more pesticides are applied to the plant can be reduced by at least about 40%. The frequency of which the one or more pesticides are applied to the plant can be reduced by at least about 50%.

Use of the compounds, salts, solvates, can allow a reduction in the amount of water applied to the plants. The amount of the water applied to the plant may be reduced by at least about 1%, 5%, 10%, 15%, 20%, 25%, 30%, 35%, 40%, 50%, 60%, 70%, 80%, 90%, 95%, or 100%. The amount of the water applied to the plant may be reduced by at least about 10%. The amount of the water applied to the plant may be reduced by at least about 20%. The amount of the water applied to the plant may be reduced by at least about 30%. The amount of the water applied to the plant may be reduced by at least about 50%.

Use of the compounds, salts, solvates, or compositions can allow a reduction in the frequency of which the water is applied to the plant. The frequency of which the water is applied to the plant can be reduced by at least about 1%, 5%, 10%, 15%, 20%, 25%, 30%, 35%, 40%, 50%, 60%, 70%, 80%, 90%, 95%, or 100%. The frequency of which the water is applied to the plant can be reduced by at least about 10%. The frequency of which the water is applied to the plant can be reduced by at least about 20%. The frequency of which the water is applied to the plant can be reduced by at least about 30%. The frequency of which the water is applied to the plant can be reduced by at least about 40%. The frequency of which the water is applied to the plant can be reduced by at least about 50%.

The compound, salt, solvate, composition disclosed herein can be used to control phytopathogenic fungi. Improving agriculture can comprise controlling unwanted plant growth. Controlling unwanted plant growth can comprise stimulating germination activity of the unwanted plant. The unwanted plant can be a parasitic plant. The unwanted plant can be a root parasitic plant. Examples of parasitic plants can include, but are not limited to, witchweeds (Striga spp.), broomrapes (Orobanche spp, Phelipanche spp), Alectra, dodders, and mistletoes. The unwanted plant can belong to the family Orobanchaceae. The unwanted plant can be witchweed. The unwanted plant can be Orobanche spp. The compound, salt, solvate, or formulation can be applied directly to the unwanted plant. The compound, salt, solvate, or formulation can be applied indirectly to the unwanted plant.

The compound, salt, solvate, or composition disclosed herein can be used to control unwanted insect or mite infestation. Examples of insects and mites can include, but are not limited to spiders, gnats, mealybugs, whiteflies, predator mites, spider mites and aphids.

The compound, salt, solvate, or composition disclosed herein can be used to regulate growth of the plant. Regulating plant growth can comprise regulating plant breeding. Regulating plant growth can comprise inhibiting shoot branching. Regulating plant growth can comprise regulating one or more plant products. Regulating plant growth can comprise inhibiting root development.

The compound, salt, solvate, or composition disclosed herein can be used to promote or stimulate activity in fungi. The compound, salt, solvate, or formulation can stimulate hyphal branching activity of one or more fungi. The compound, salt, solvate, or formulation can induce spore germination of one or more fungi. The one or more fungi can be arbuscular mycorrhizal (AM) fungi.

Further disclosed herein are methods of preserving or extending the life of a plant. Generally, the method can comprise contacting the plant (e.g. a leaf, stem, root, or any part of a plant) with a compound, salt, solvate, or composition disclosed herein. The compound, salt, solvate, or composition for use in preserving or extending the life of a plant can be produced by any of the methods disclosed herein.

The compound, salt, solvate, or composition may be used to preserve or extend the life of a cut plant. The cut plant can be a flower. The cut plant can be a tree. The cut plant can be bush or shrub. The cut plant can be a vegetable. The compound, salt, solvate, or composition can be used to preserve or extend the life of an uncut plant. The uncut plant can be a flower. The uncut plant can be a tree. The uncut plant can be bush or shrub. The uncut plant can be a vegetable. The compound, salt, solvate, or composition can be used to preserve or extend the life of a potted plant. The potted plant can be a flower. The potted plant can be a tree. The potted plant can be bush or shrub. The potted plant can be a vegetable.

The compound, salt, solvate, or composition can be used to preserve or extend the life of a flower. Examples of flowers can include, but are not limited to, lilies, daisies, roses, marigolds, Angel's trumpet, phlox, vinca, snapdragons, toadflax, orchids, ferns, black-eyed Susans, blood flowers, blue lobelias, morning glories, poppies, calendulas, geraniums, impatiens, lantanas, larkspurs, calla lilies, hyacinths, azaleas, pointsettias, and begonias.

The compound, salt, solvate, or composition can be used to preserve or extend the life of a bush or shrub. Examples of bushes and shrubs can include, but are not limited to, forsynthia, fuchsia, hibiscus, currant, lilac, rose, hydrangea, willow, magnolia, thyme, snowberry, dogwood and holly.

The compound, salt, solvate, or composition can be used to preserve or extend the life of a tree. Examples of trees can include, but are not limited to, cypress, poinsettia, palm, fir, pine, spruce, cedar, oak, mulberry, chestnut, hawthorn, poplar, and maple. The tree can be a fir tree. The fir tree can be a Douglas, Balsam or Fraser fir tree. The tree can be a pine tree. The pine tree can be a Scotch or White pine tree. The tree can be a spruce tree. The spruce tree can be a White, Norway or Blue spruce tree. The tree can be a cedar tree. The cedar tree can be a Deodara or Eastern red cedar. The tree can be a cypress tree. The cypress tree can be an Arizona or Leland cypress tree.

The plant can be contacted with a compound, salt, solvate, or composition disclosed herein, thereby extending or preserving the life of the plant. Contacting the plant with the compound, salt, solvate, or composition can comprise administering the compound, salt, solvate, or composition as a spray. Contacting the plant with the compound, salt, solvate, or composition can comprise adding the plant growth material to the irrigation water of the plant. Contacting the plant with the compound, salt, solvate, or composition can comprise applying the compound, salt, solvate, or composition to the habitat of the plant. Contacting the plant with the compound, salt, solvate, or composition can comprise adding the compound, salt, solvate, or composition to a plant container (e.g., vase) and placing the plant in the plant container. Contacting the plant with the compound, salt, solvate, or composition can comprise adding the compound, salt, solvate, or composition to soil.

The life of the plant can be extended by at least about 1%, 5%, 10%, 15%, 20%, 25%, 30%, 35%, 40%, 45%, 50%, 55%, 60%, 65%, 70%, 75%, 80%, 85%, 90%, 95%, or 97% as compared to an untreated plant. The life of the plant can be extended by at least about 20% as compared to an untreated plant. The life of the plant can be extended by at least about 30% as compared to an untreated plant. The life of the plant can be extended by at least about 40% as compared to an untreated plant. The life of the plant can be extended by at least about 50% as compared to an untreated plant. The life of the plant can be extended by at least about 55% as compared to an untreated plant. The life of the plant can be extended by at least about 60% as compared to an untreated plant. The life of the plant can be extended by at least about 65% as compared to an untreated plant. The life of the plant can be extended by at least about 70% as compared to an untreated plant. The life of the plant can be extended by at least about 75% as compared to an untreated plant. The life of the plant can be extended by at least about 80% as compared to an untreated plant. The life of the plant can be determined by measuring the growth time between initial planting of a seed of the plant to the death of the plant.

The life of the plant can be extended by at least about 6, 12, 24, 30, 36, 42, 48, 54, 60, 66, 72, 78, 84, 90, 96, 102, 108, 114, or 120 hours as compared to an untreated plant. The life of the plant can be extended by at least about 24 hours as compared to an untreated plant. The life of the plant can be extended by at least about 36 hours as compared to an untreated plant. The life of the plant can be extended by at least about 48 hours as compared to an untreated plant. The life of the plant can be extended by at least about 72 hours as compared to an untreated plant. The life of the plant can be extended by at least about 96 hours as compared to an untreated plant.

The life of the plant can be extended by at least about 1, 1.5, 2, 2.5, 3, 3.5, 4, 4.5, 5, 5.5, 6, 6.5, or 7 days as compared to an untreated plant. The life of the plant can be extended by at least about 8, 9, 10, 11, 12, 13, 14, 15, 16, 17, 18, 19, or 20 days as compared to an untreated plant. The life of the plant can be extended by at least about 1 day as compared to an untreated plant. The life of the plant can be extended by at least about 2 days as compared to an untreated plant. The life of the plant can be extended by at least about 2.5 days as compared to an untreated plant. The life of the plant can be extended by at least about 3 days as compared to an untreated plant. The life of the plant can be extended by at least about 3.5 days as compared to an untreated plant. The life of the plant can be extended by at least about 4 days as compared to an untreated plant. The life of the plant can be extended by at least about 4.5 days as compared to an untreated plant.

The life of the plant can be extended by at least about 1, 1.5, 2, 2.5, 3, 3.5, 4, 4.5, 5, 5.5, 6, 6.5, or 7 weeks as compared to an untreated plant. The life of the plant can be extended by at least about 8, 9, 10, 11, 12, 13, 14, 15, 16, 17, 18, 19, or 20 weeks as compared to an untreated plant. The life of the plant can be extended by at least about 1, 1.5, 2, 2.5, 3, 3.5, 4, 4.5, 5, 5.5, 6, 6.5, or 7 months as compared to an untreated plant. The life of the plant can be extended by at least about 8, 9, 10, 11, 12, 13, 14, 15, 16, 17, 18, 19, or 20 months as compared to an untreated plant.

Preserving or extending the life of the plant can comprise reducing wilting of the plant. Reducing wilting of the plant can comprise reducing flower or leaf rolling of the plant. The wilting of the plant can be reduced by at least about 1%, 5%, 10%, 15%, 20%, 25%, 30%, 35%, 40%, 45%, 50%, 55%, 60%, 65%, 70%, 75%, 80%, 85%, 90%, 95%, or 97% as compared to an untreated plant. The wilting of the plant can be reduced by at least about 10% as compared to an untreated plant. The wilting of the plant can be reduced by at least about 30% as compared to an untreated plant. The wilting of the plant can be reduced by at least about 50% as compared to an untreated plant. The wilting of the plant can be reduced by at least about 70% as compared to an untreated plant. The wilting of the plant can be reduced by at least about 80% as compared to an untreated plant.

A sign of plant stress can include wilting of the plant. For example, stressed plants can have rolled leaves or petals. The plant growth materials disclosed herein can promote the life of the plant by reducing the wilting of the plant. Reducing the wilting of the plant can comprise delaying the wilting of the plant as compared to an untreated plant. For example, an untreated cut plant can show signs of wilting within 36 hours of being cut, however, a cut plant treated with a plant growth material can have delayed wilting. The wilting of the plant can be delayed by at least about 2, 3, 4, 5, 6, 7, 8, 9, 10, 11, 12, 13, 14, 15, 16, 17, 18, 19, 20, 21, 22, 23, or 24 hours as compared to an untreated plant. The wilting of the plant can be delayed by at least about 12 hours as compared to an untreated plant. The wilting of the plant can be delayed by at least about 24 hours as compared to an untreated plant. The wilting of the plant can be delayed by at least about 36 hours as compared to an untreated plant. The wilting of the plant can be delayed by at least about 48 hours as compared to an untreated plant.

An additional sign of plant stress can include reduced turgidity. Turgidity can refer to pressure caused by the osmotic flow of water from an area of low solute concentration outside of the cell into the cell cell's vacuole. Turgidity can be used by plants to maintain rigidity. Often, healthy plants are turgid, whereas, unhealthy plants are less turgid. Preserving or extending the life of the plant can comprise prolonging or maintaining the turgidity of the plant. The turgidity of the plant can be greater than the turgidity of an untreated plant. The turgidity of the plant can be at least about 1%, 5%, 10%, 15%, 20%, 25%, 30%, 35%, 40%, 45%, 50%, 55%, 60%, 65%, 70%, 75%, 80%, 85%, 90%, 95%, or 97% greater than the turgidity of an untreated plant. The turgidity of the plant can be at least about 10% greater than the turgidity of an untreated plant. The turgidity of the plant can be at least about 15% greater than the turgidity of an untreated plant. The turgidity of the plant can be at least about 25% greater than the turgidity of an untreated plant. The turgidity of the plant can be at least about 35% greater than the turgidity of an untreated plant. The turgidity of the plant can be at least about 45% greater than the turgidity of an untreated plant. The turgidity of the plant can be at least about 60% greater than the turgidity of an untreated plant. The turgidity of the plant can be at least about 75% greater than the turgidity of an untreated plant.

A stressed plant can also show a reduction in the turgid state. The turgid state can refer to a period of time in which the plant maintains its rigidity. The rigidity of the plant can refer to the rigidity of the stem of the plant. For example, as cut plants die, the stem of the plant can be less rigid, thereby causing the cut plant to fall over or bend. A stressed plant can be unable to hold itself upright. Preserving or extending the life of the plant can comprise prolonging the turgid state of the plant. The turgid state of the plant can be increased by at least about 1%, 5%, 10%, 15%, 20%, 25%, 30%, 35%, 40%, 45%, 50%, 55%, 60%, 65%, 70%, 75%, 80%, 85%, 90%, 95%, or 97% as compared to an untreated plant. The turgid state of the plant can be increased by at least about 20% as compared to an untreated plant. The turgid state of the plant can be increased by at least about 30% as compared to an untreated plant. The turgid state of the plant can be increased by at least about 40% as compared to an untreated plant. The turgid state of the plant can be increased by at least about 50% as compared to an untreated plant.

The turgid state of the plant can be increased by at least about 2, 3, 4, 5, 6, 7, 8, 9, 10, 11, 12, 13, 14, 15, 16, 17, 18, 19, 20, 21, 22, 23, or 24 hours as compared to an untreated plant. The turgid state of the plant can be increased by at least about 6 hours as compared to an untreated plant. The turgid state of the plant can be increased by at least about 12 hours as compared to an untreated plant. The turgid state of the plant can be increased by at least about 24 hours as compared to an untreated plant.

A stressed plant can lose leaves or petals. Contacting a plant with a plant growth material can reduce or delay the loss of one or more petals or leaves of the plant. For example, an untreated plant can lose 50% of its leaves or petals, whereas a treated plant can lose 10-25% of its leaves or petals. The loss of the one or more petals of the plant can be reduced by least about 1%, 5%, 10%, 15%, 20%, 25%, 30%, 35%, 40%, 45%, 50%, 55%, 60%, 65%, 70%, 75%, 80%, 85%, 90%, 95%, or 97% as compared to the loss of the one or more petals of an untreated plant. The loss of the one or more petals of the plant can be reduced by least about 10% as compared to the loss of the one or more petals of an untreated plant. The loss of the one or more petals of the plant can be reduced by least about 20% as compared to the loss of the one or more petals of an untreated plant. The loss of the one or more petals of the plant can be reduced by least about 35% as compared to the loss of the one or more petals of an untreated plant. The loss of the one or more petals of the plant can be reduced by least about 50% as compared to the loss of the one or more petals of an untreated plant. The loss of the one or more petals of the plant can be reduced by least about 60% as compared to the loss of the one or more petals of an untreated plant. The loss of the one or more petals of the plant can be reduced by least about 70% as compared to the loss of the one or more petals of an untreated plant.

The loss of the one or more petals of the plant can be delayed by at least about 2, 3, 4, 5, 6, 7, 8, 9, 10, 11, 12, 13, 14, 15, 16, 17, 18, 19, 20, 21, 22, 23, or 24 hours as compared to the loss of one or more petals of an untreated plant. The loss of the one or more petals of the plant can be delayed by at least about 25, 30, 35, 40, 45, 50, 55, 60, 65, 70, 75, 80, 85, 90, 95 or 100 hours as compared to the loss of one or more petals of an untreated plant. The loss of the one or more petals of the plant can be delayed by at least about 6 hours as compared to the loss of one or more petals of an untreated plant. The loss of the one or more petals of the plant can be delayed by at least about 12 hours as compared to the loss of one or more petals of an untreated plant. The loss of the one or more petals of the plant can be delayed by at least about 18 hours as compared to the loss of one or more petals of an untreated plant. The loss of the one or more petals of the plant can be delayed by at least about 36 hours as compared to the loss of one or more petals of an untreated plant. The loss of the one or more petals of the plant can be delayed by at least about 48 hours as compared to the loss of one or more petals of an untreated plant. The loss of the one or more petals of the plant can be delayed by at least about 60 hours as compared to the loss of one or more petals of an untreated plant. The loss of the one or more petals of the plant can be delayed by at least about 72 hours as compared to the loss of one or more petals of an untreated plant. The loss of the one or more petals of the plant can be delayed by at least about 96 hours as compared to the loss of one or more petals of an untreated plant.

A stressed plant can show signs of discoloration. The stressed plant can appear brownish. Alternatively, or additionally, the stressed plant shows a reduction in the appearance of green leaves. The chlorophyll content of the stressed plant can also be reduced. Preserving or extending the life of the plant can comprise maintaining the chlorophyll content of the plant. For example, a reduction in the chlorophyll content of an untreated plant can appear within 48 hours of being cut. However, a reduction in the chlorophyll content of a treated plant can appear after 60 hours of being cut. The chlorophyll content of the plant can be maintained for at least about 2, 3, 4, 5, 6, 7, 8, 9, 10, 11, 12, 13, 14, 15, 16, 17, 18, 19, 20, 21, 22, 23, or 24 hours. The chlorophyll content of the plant can be maintained for at least about 6 hours. The chlorophyll content of the plant can be maintained for at least about 12 hours. The chlorophyll content of the plant can be maintained for at least about 24 hours. Discoloration such as leaf firing (premature yellowing) can occur as a result of poor nutrient availability, and can be an indicator of poor plant health. For, example, leaf firing can be a result of nitrogen deficiency.

Preserving or extending the life of the plant can comprise reducing or delaying the loss of the chlorophyll content of the plant. The chlorophyll content of the plant can be greater than the chlorophyll content of an untreated plant. The chlorophyll content of the plant can be at least about 1%, 5%, 10%, 15%, 20%, 25%, 30%, 35%, 40%, 45%, or 50% greater than the content of an untreated plant. The chlorophyll content of the plant can be at least about 55%, 60%, 65%, 70%, 75%, 80%, 85%, 90%, 95% or 97% greater than the content of an untreated plant. The chlorophyll content of the plant can be at least about 20% greater than the content of an untreated plant. The chlorophyll content of the plant can be at least about 30% greater than the content of an untreated plant. The chlorophyll content of the plant can be at least about 40% greater than the content of an untreated plant. The chlorophyll content of the plant can be at least about 50% greater than the content of an untreated plant. The chlorophyll content of the plant can be at least about 60% greater than the content of an untreated plant. The chlorophyll content of the plant can be at least about 1.5, 2, 2.5, 3, 3.5, 4, 4.5, 5, 5.5, 6, 6.5, 7, 8, 9, or 10-fold greater than the content of an untreated plant. The chlorophyll content of the plant can be at least about 11, 12, 13, 14, 15, 16, 17, 18, 19, 20, 25, 30, 35, 40, 45, 50, 55, 60, 65, 70, 75, 80, 85, 90, 95 or 100-fold greater than the content of an untreated plant. The chlorophyll content of the plant can be at least about 2-fold greater than the content of an untreated plant. The chlorophyll content of the plant can be at least about 3-fold greater than the content of an untreated plant. The chlorophyll content of the plant can be at least about 4-fold greater than the content of an untreated plant. The chlorophyll content of the plant can be at least about 5-fold greater than the content of an untreated plant. The chlorophyll content of the plant can be at least about 10-fold greater than the content of an untreated plant.

The loss of the chlorophyll content of the plant can be delayed by at least about 2, 3, 4, 5, 6, 7, 8, 9, 10, 11, 12, 13, 14, 15, 16, 17, 18, 19, 20, 21, 22, 23, or 24 hours as compared to the loss of the chlorophyll content of an untreated plant. The loss of the chlorophyll content of the plant can be delayed by at least about 30, 35, 40, 45, 50, 55, 60, 65, 70, 75, 80, 85, 90, 95, or 100 hours as compared to the loss of the chlorophyll content of an untreated plant. The loss of the chlorophyll content of the plant can be delayed by at least about 6 hours as compared to the loss of the chlorophyll content of an untreated plant.

The loss of the chlorophyll content of the plant can be delayed by at least about 12 hours as compared to the loss of the chlorophyll content of an untreated plant. The loss of the chlorophyll content of the plant can be delayed by at least about 24 hours as compared to the loss of the chlorophyll content of an untreated plant. The loss of the chlorophyll content of the plant can be delayed by at least about 36 hours as compared to the loss of the chlorophyll content of an untreated plant. The loss of the chlorophyll content of the plant can be delayed by at least about 48 hours as compared to the loss of the chlorophyll content of an untreated plant. The loss of the chlorophyll content of the plant can be delayed by at least about 60 hours as compared to the loss of the chlorophyll content of an untreated plant. The loss of the chlorophyll content of the plant can be delayed by at least about 72 hours as compared to the loss of the chlorophyll content of an untreated plant.

The loss of the chlorophyll content of the plant can be less than the loss of the chlorophyll content of an untreated plant. The loss of the chlorophyll content of the plant can be at least about 1%, 5%, 10%, 15%, 20%, 25%, 30%, 35%, 40%, 45%, 50%, 55%, or 60% less than the loss of the chlorophyll content of an untreated plant. The loss of the chlorophyll content of the plant can be at least about 65%, 70%, 72%, 75%, 77%, 80%, 85%, 90%, 92%, 95%, or 97% less than the loss of the chlorophyll content of an untreated plant. The loss of the chlorophyll content of the plant can be at least about 5% less than the loss of the chlorophyll content of an untreated plant. The loss of the chlorophyll content of the plant can be at least about 10% less than the loss of the chlorophyll content of an untreated plant. The loss of the chlorophyll content of the plant can be at least about 20% less than the loss of the chlorophyll content of an untreated plant. The loss of the chlorophyll content of the plant can be at least about 30% less than the loss of the chlorophyll content of an untreated plant. The loss of the chlorophyll content of the plant can be at least about 40% less than the loss of the chlorophyll content of an untreated plant. The loss of the chlorophyll content of the plant can be at least about 50% less than the loss of the chlorophyll content of an untreated plant. The loss of the chlorophyll content of the plant can be at least about 60% less than the loss of the chlorophyll content of an untreated plant.

The loss of the chlorophyll content of the plant can be at least about 1.5, 2, 2.5, 3, 3.5, 4, 4.5, 5, 5.5, 6, 6.5, 7, 7.5, 8, 8.5, 9, 9.5, or 10-fold less than the loss of the chlorophyll content of an untreated plant. The loss of the chlorophyll content of the plant can be at least about 11, 12, 13, 14, 15, 16, 17, 18, 19, 20, 25, 30, 35, 40, 45, 50, 55, 60, 65, 70, 75, 80, 85, 90, 95 or 100-fold less than the loss of the chlorophyll content of an untreated plant. The loss of the chlorophyll content of the plant can be at least about 2-fold less than the loss of the chlorophyll content of an untreated plant. The loss of the chlorophyll content of the plant can be at least about 3-fold less than the loss of the chlorophyll content of an untreated plant. The loss of the chlorophyll content of the plant can be at least about 5-fold less than the loss of the chlorophyll content of an untreated plant. The loss of the chlorophyll content of the plant can be at least about 10-fold less than the loss of the chlorophyll content of an untreated plant.

The compound, salt, solvate, or composition can be applied directly to the plant. The compound, salt, solvate, or composition can be applied to one or more parts of the plant. The one or more parts of the plant can comprise a terminal bud, flower, lateral bud, leaf blade, leaf axil, node, internode, petiole, primary root, lateral root, root hair, root cap, or a combination thereof. The composition can be applied to the leaf blade of the plant. The compositions can be applied to the root of the plant.

Alternatively, or additionally, the compound, salt, solvate, or composition can be applied to a soil. The composition can be applied to an area around the plant. The area around the plant can comprise soil. The area around the plant can comprise an adjacent plant. The composition can be applied to a soil before placing a plant or seed in the soil. The composition can be applied to bacterial consortium present in the soil. The composition can be applied with additional bacteria to supplement the natural bacterial consortium in the soil.

The compound, salt, solvate, or composition can be applied to a plant that is susceptible to a parasitic weed. Examples of plants include, but are not limited to, corn, rice, sorghum, millets, and sugar cane. The plant can be corn. The plant can be tobacco. The plant can be rice.

The compound, salt, solvate, or composition can be applied as a seed coating. The compound, salt, solvate, or composition can be applied as a seed treatment. The compound, salt, solvate, or composition can be applied as a seed dressing. The compound, salt, solvate, or composition can be applied as a spray. The compound, salt, solvate, or composition can be applied as a foliar spray. The compound, salt, solvate, or composition can be applied as a powder. The powder can be a wettable powder.

The compound, salt, solvate, or composition can be applied 1, 2, 3, 4, 5, 6, 7, 8, 9, 10 or more times a day. The compound, salt, solvate, or composition can be applied once a day. The compound, salt, solvate, or composition can be applied twice a day. The compound, salt, solvate, or composition can be applied 1, 2, 3, 4, 5, 6, 7, 8, 9, 10 or more times per week. The compound, salt, solvate, or composition can be applied once a week. The compound, salt, solvate, or composition can be applied twice a week. The compound, salt, solvate, or composition can be applied three times a week. The compound, salt, solvate, or composition can be applied four times a week. The compositions can be applied 1, 2, 3, 4, 5, 6, 7, 8, 9, 10 or more times a month. The compositions can be applied once a month. The compound, salt, solvate, or composition can be applied twice a month. The compound, salt, solvate, or composition can be applied three times a month. The compound, salt, solvate, or composition can be applied four times a month. The compositions can be applied ten times a month. The compound, salt, solvate, or composition can be applied 15 times a month. The compositions can be applied 20 times a month.

In some instances, the measurement described herein can be made at a temperature of about 1, 2, 3, 4, 5, 6, 7, 8, 9, 10, 11, 12, 13, 14, 15, 16, 17, 18, 19, 20, 21, 22, 23, 24, 25, 6, 27, 28, 29, 30, 31, 32, 33, 34, 35, 36, 37, 38, 39, or 40° C.

### EXAMPLES

### Example 1. A Test Compound Stimulates Phosphate Solubilization Activity of a Pure Isolate of Soil Bacterium

A 5 mL culture of B. megaterium was seeded from a single colony into Nutrient Broth (NB) and grown overnight in a 30° C. shaker. The cell pellet was collected by centrifugation, washed twice and resuspended in water. The concentration of B. megaterium was measured using a Nanodrop OD₆₀₀ reading. B. megaterium was inoculated into liquid NBRIP media, which contains insoluble tricalcium phosphate [53 mM] Ca₃(PO₄)₂) as its sole phosphorus source. The final concentration of B. megaterium in the NBRIP media was OD₆₀₀=0.02 (3×10³ CFU/mL). NBRIP media was supplemented with a test compound in a 1% DMSO solution to a final concentration of 100 μg/mL. The compound and DMSO were filter sterilized through a 0.2 μM filter.

At the experiment start and after 72 hours of growth, 1 mL of culture was collected from the culture tubes. The supernatant was collected by centrifugation (5 min at 13,000 rpm). The cleared supernatant was diluted 1:00 in molecular grade water and used for orthophosphate analysis with the malachite-green method. The remaining 4 mL of supernatant were collected by centrifugation and used for pH readings.

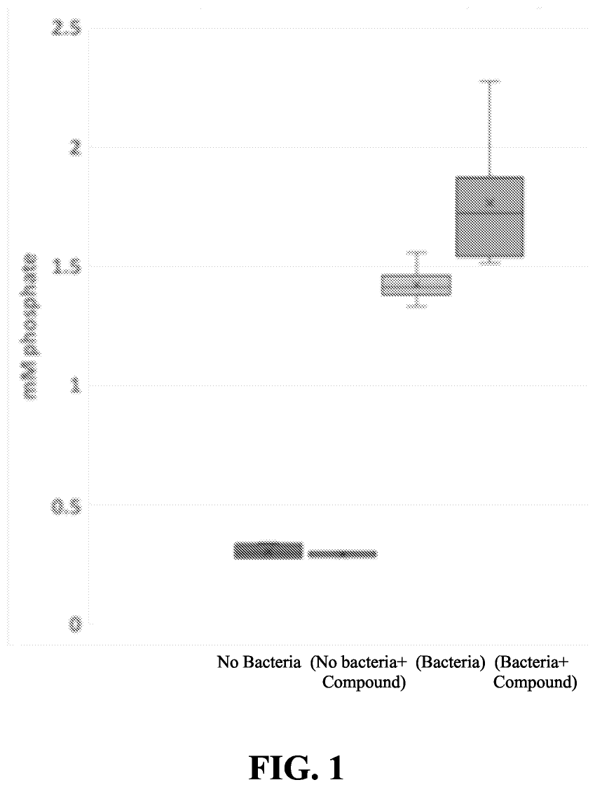

FIG. 1 illustrates that an exemplary compound, quercetin (QC), stimulates production of soluble orthophosphate when contacted with the B. megaterium reporter strain. Indeed, contacting the bacteria with QC significantly increases the concentration of soluble orthophosphate, relative to bacteria not contacted with QC.

### Example 2. A Test Compound Stimulates Phosphate Solubilization in Soil Consortia

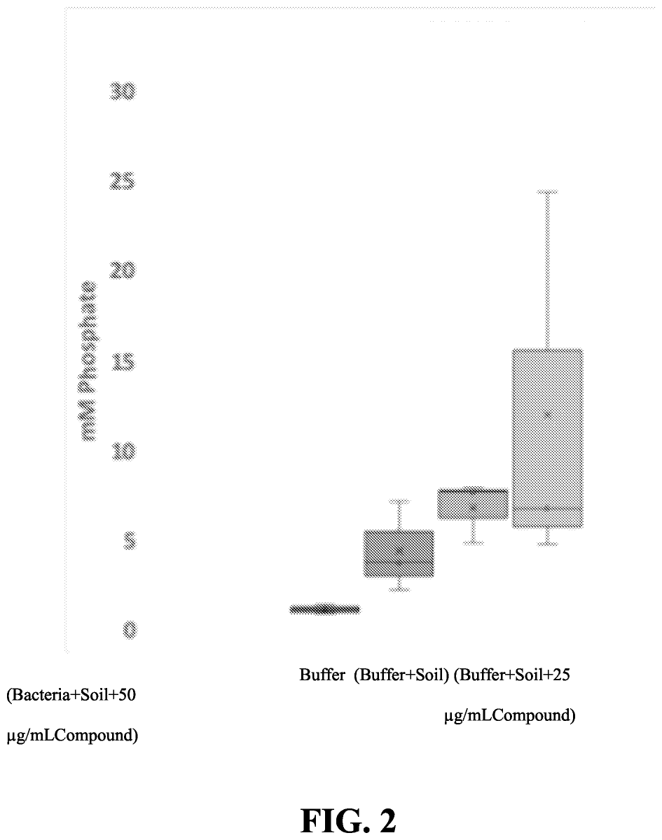

To determine if a test compound, QC, alters phosphate solubilization in a soil's native microbial community, dried and sieved soil was used as a bacterial inoculum in NBRIP media with and without QC. As shown in FIG. 2, treatment increased the amount of detectable orthophosphate in the growth media of soil-inoculated cultures, relative to soil that was not contacted with QC. Further, the amount of stimulation appears to be dose dependent, as 50 μg/mL QC appears to increase phosphate solubilization to a greater extent than 25 μg/mL QC.

### Example 3. A Test Compound Induces Nitrogen Fixing Gene Cluster

Three hundred base pairs upstream of the ATG of the nifHDK of Azotobacter vinelandii (nifHipro) was cloned in frame, upstream of luciferase in pVSP61 (kanR). Azotobacter vinelandii (Lipman ATCC® BAA-1303) was transformed with the reporter plasmid with triparental mating, Transformants were selected for on nitrogen-free kanamycin plates and confirmed with PCR and sequencing.

Five milliliters of nifHpro::luciferase Bioreporter strain #171 was grown overnight in autoclaved, nitrogen-free medium (Burks, HiMedia M707) at starting OD₆₀₀=0.02 with 1 ug/mL Kanamycin in sterile 50 mL flasks. Chemical treatments were dissolved in 100% EtOH and added at appropriate concentrations, keeping the volume constant. The solvent was no more than 2% of the final culture volume, and solvent only controls were included in each experiment.

After 24 h, the growth density each culture was read in a clear, flat-bottomed microplate by adding 180 μl to each well (flasks were measured in triplicate) and measuring absorbance at 600 nm in a microplate reader. The samples were transferred to a black, opaque bottomed microplate and 20 μl of 10 mM Luciferin (Thermo Scientific, Cat No. 88294) was added for a final concentration of 1 mM. Samples were mixed well by pipetting and allowed to incubate at room temperature for 10 minutes.

The luminescence of each well was read on an iD3 plate reader (Molecular Devices). Luminescence of each well (reported as RLU, Relative Light Unit) was divided by its OD₆₀₀ in order to adjust for culture density.

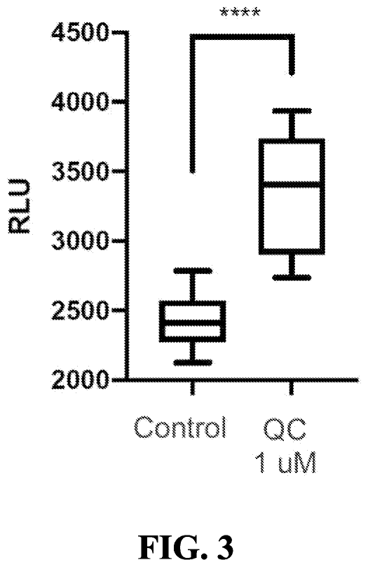

As shown in FIG. 3, incubation of the Azotobacter vinelandii reporter bacteria with a test compound, QC, significantly increases the amount of nitrogen fixation, relative to control bacteria not contacted with QC. Specifically, contacting the bacteria strain with 1 μM of QC produced a significant increase in luminescence, relative to the control bacteria not contacted with QC, thereby indicating a significant increase in nitrogen fixation due to QC.

### Example 4. Treatment of Plants with Compounds Described Herein Produces Healthier Plants

Seed Treatments and Planting

Various compounds of formulas described herein were screened for their ability to produce increases in plant biomass. Active ingredient solutions were made at 0.1 mM by dissolving the compound into acetone. An acetone only dose with no active ingredient (0 mM) was used as a control. The final volume of each dose solution was 3.75 ml, which was placed in a glass scintillation vial containing ˜360 wheat seeds (variety Patwin) (UC Davis Foundation Seed Program). The seeds with treatment solution were vigorously mixed and allowed to dry for 24 hours.

The treated and control seeds were planted in standard planting inserts which were held in 8-ounce meal prep trays (Ez Prepa™) (6 inserts/tray) to allow for bottom watering. The growth medium used was Turface™ (Profile Products LLC, Buffalo Grove, TL) which is an inert calcinated clay. Each insert was fill with ˜40 ml of Turface™. A single cell insert (2.35″×2.15″×2.33″) made up for one experimental representative, in which ˜48 seeds were planted. Planting was done by uniformly spreading seeds on top of the growth medium, following moistening with a water mist from a spray bottle. The inserts were then bottom watering with 250 ml of water, covered with foil, and incubated for 48 hr in darkness. The foil was then removed and the trays containing the inserts were placed under grow lights for a 1-week growth period. Bottom watering was done daily by adding 100 ml of water. The seedlings were initially monitored for uniform seed germination and fungal contamination. Each compound was tested using a dose curve of 0.001 mM, 0.01 mM, 0.1 mM, 1 mM and 10 mM at 6 reps each and a corresponding control (0 mM) with 12 reps. All experimental reps and controls were randomized under the grow lights, and their positions changed every three days.

Plant Phenotype Analysis for Compound Assessment

Plants were grown for three weeks in a grow room under a light/dark (L/D) cycle of 16 h/L at 24° C. and 8 h/D at 20° C. using LED lights (Next Light™, Cincinnati, Ohio) with a light intensity of 365 PPF (μmol/sec).

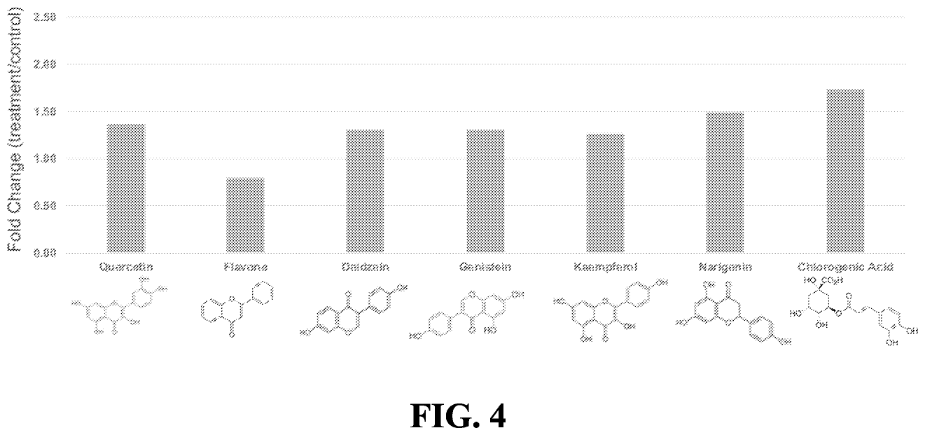

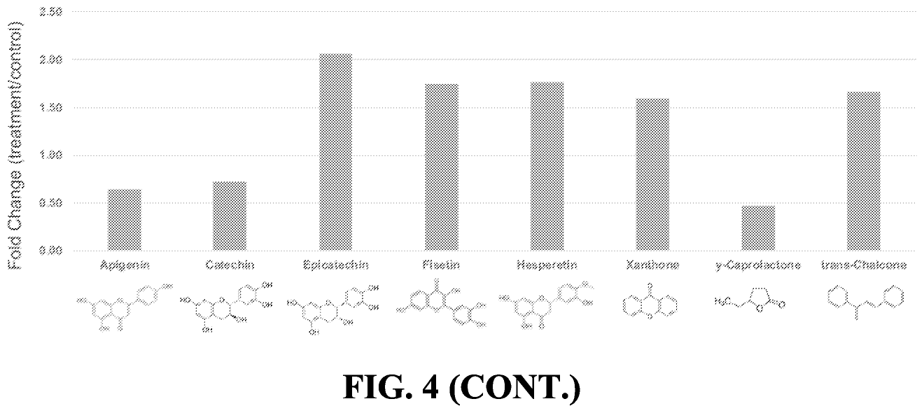

FIG. 4 depicts the fold change in plant biomass among plants contacted with the compounds tested, relative to control plants that were contacted with control solutions lacking the compounds. As shown in Figure, several compounds were able to significantly increase the biomass of the plant relative to control, with increases ranging from about 1 fold to about two fold, relative to control plants.

While exemplary embodiments have been shown and described herein, such embodiments are by way of example only. Numerous variations, changes, and substitutions can be performed on the exemplary embodiments. It should be understood that various alternatives to the embodiments described herein may be employed.

### Example 5. Stimulation of Phosphate Solubilization Activity of a Pure Isolate of Soil Bacterium (Bacillus megaterium)

Bacillus megaterium is a common soil bacteria known to have phosphate solubilization activity. A strain of B. megaterium was isolated from Iowa field soil and its ability to solubilize phosphorus was confirmed by observing a cleared zone (or halo) around B. megaterium colonies when grown on solid media (NBRIP+Agar) containing only in insoluble form of phosphorus, Ca₃(PO₄)₂.

Cultures of B. megaterium were then grown in liquid NBRIP (containing only insoluble Ca₃(PO₄)₂) with and without Formula Id. The growth media supernatant was analyzed for orthophosphate at 4 days after growth by the malachite green quantification method.

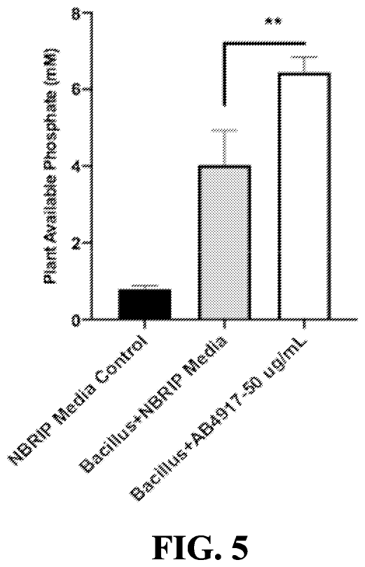

A statistically significant increase in phosphate solubilization in B. megaterium cultures treated with Formula Id was observed 4 days post treatment, compared to untreated cultures (FIG. 5).

### Example 6. Stimulation of nifHpro in a Model Nitrogen Fixing Free Living Soil Diazotroph (Azotobacter vinelandii)

Creating the nfHpro::Luciferase Azotobacter vinelandii Reporter Line

Gibson Assembly was used to clone the luciferase gene (ordered from IDT) into pE_Gm and LR into pVSP61 (plasmids provided by Doug Dahlbeck at Staskawicz Lab, UC Berkeley). Standard triparental mating was used with pRK600 (provided by Doug Dahlbeck at Staskawicz Lab, UC Berkeley) to obtain the final reporter strain of Azotobacter vinelandii transformed with reporter plasmid.

Assaying Nitrogenase (nifH) Activity with rufHpro::Luciferase Azotobacter vinelandii Reporter Line

nifHpro::luciferase Azotobacter vinelandii were grown for 24 hours in 50 mL liquid Burks —N media (HiMedia Laboratories) with 1 ng/μL Kanamycin, shaking at 100 RPM under lights at ˜30° C. After 24 hours, supernatant was spun down 12000 G for two minutes to collect cells. After supernatant was removed, cells were washed with cleared Burks —N liquid media supernatant, resuspended, and centrifuged again at 12000 G. This process was repeated, for a total of two washes.

Resuspended cells were combined with Burks —N media with 1 ng/μL Kanamycin, to obtain a fresh culture at OD 0.1. 50 mL shake flasks were then inoculated with 5 mL of this culture, plus appropriate chemistry, in the scheme that follows. A stock solution of Quercetin was made in DMSO. 10 biological replicates of 10 μM, 5 μM, 1 μM, 0.1 μM and 0.001 μM Formula Id in DMSO, plus 10 control replicates, were prepared in the 50 mL shake flasks. The flasks were secured and shaken at 100 RPM under lights at ˜30° C. for 21 hours.

After 21 hours, 50 mL flasks were removed from the shaker. Supernatant samples were taken from each flask and normalized to OD 0.3. To measure activity of nifH/luciferase, three technical replicates (180 μL each) from each sample were plated on a black round-bottom 96 well plate and incubated with 20 μL 10 mM luciferin for 5 minutes. Plates were inserted into plate reader and luminescence of each well was analyzed. Following collection of this data, the concentration—luminescence response graph was generated using Prism graph software.

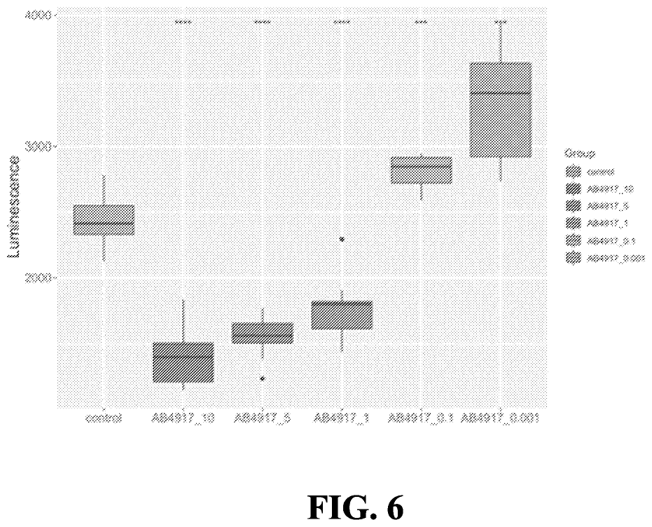

In repeated laboratory experiments (n=9), Formula Id activated the nifHpro::luciferase bioreporter over controls, indicating an increase in nitrogenase gene expression in Azotobacter vinelandii, a free-living nitrogen fixing bacteria (FIG. 6).

### Example 7. Stimulation of Microbial Phosphate Solubilization when Applied as a Foliar Spray in Corn

In order to test the ability of Formula Id to stimulate a phosphate solubilization effect when applied on plants versus directly into soil, a 3 week old corn plant was introduced into field soil consortia cultures.

Approximately two weeks prior to the experiment, corn was removed from soil and roots were cleaned of potting soil. Plants were left in tap water to induce nutrient stress and showed purple streaking, a phenotypic sign associated with phosphate starvation. Corn leaves were sprayed with Formula Id (roots protected with foil during spray application) and roots were placed in flasks of NBRIP liquid media and soil inoculant. The flasks were placed on a low speed orbital shaker under fluorescent lights and the media supernatant was tested for phosphate concentration after 24 h.

B73 Corn plants were grown until V3 growth stage, removed from potting soil, rinsed, and placed in tap water for 1.5 weeks to induce nutrient stress. Plants received foliar (3 mL/plant using a fingertip sprayer) applied treatments and were placed in 250 mL baffled flasks containing 50 mL NBRIP growth medium ([53 mM] Ca3(PO₄)₂) and 500 mg of 2 mm particle-sized field soil. Flasks with treated corn and sterilized foam caps were placed on orbital shakers at 100 RPM for 1 day at room temperature under fluorescent lights. Orthophosphate was measured using the malachite-green phosphate method.

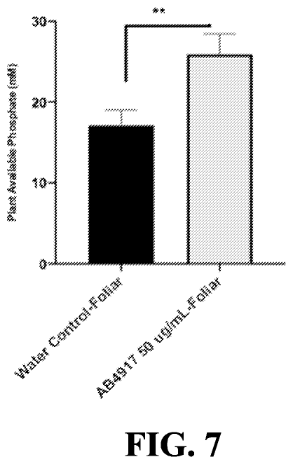

As shown in FIG. 7, a statistically significant increase in phosphate solubilization was observed in response to Formula Id treatment applied to corn leaves as a foliar spray when compared to water control.

1. A liquid composition that comprises:
(a) a compound or salt thereof of Formula I: 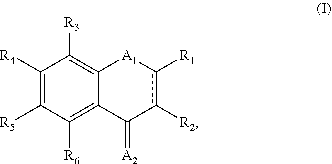
wherein:
A¹ and A² are independently O or S;
R₁ and R₂ are independently —H, —OH, —COOH, —SH, C₁-C₆ alkyl, C₃-C₆ cycloalkyl, or —Xₚ, wherein —Xₚ is: 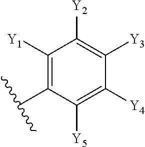
wherein Y₁, Y₂, Y₃, Y₄, and Y₅ are independently —H, —OH, —SH, —F, —Cl, —Br, —I, or —O—Z₁, wherein Z₁ is C₁-C₄ alkyl; or
wherein R₁ and R₂ along with the carbon atoms connecting them form a five or six-membered cycloalkyl ring or cycloalkenyl ring, or a five or six-membered aryl ring; and
R₃, R₄, R₅, and R₆ are independently —H, —OH, —F, —Cl, —Br, —I, or —SH; and
(b) an excipient, diluent, or carrier;
wherein the liquid composition comprises an amount of the compound or salt thereof that is at least partially effective to produce:
(a) an increased level of soluble orthophosphate of at least about 20% after contacting the amount of the compound or salt thereof with a live microbe, optionally a live Bacillus megaterium bacteria strain, relative to a level of the soluble orthophosphate produced by the live microbe prior to the contacting, as determined by an in vitro assay comprising:
(i) incubating the live microbe at an optical density at 600 nm (OD₆₀₀) of 0.02 with tricalcium phosphate at a final concentration of about 50 mM;
(ii) collecting a sample of a liquid culture from the live microbe 72 hours after the incubating; and
(iii) quantifying the level of the orthophosphate in the liquid culture using a malachite-green method; or
(b) an increased level of nitrogen fixation after contacting the amount of the compound or salt thereof with the live microbe or a reporter bacteria strain, optionally a reporter Azotobacter vinelandii bacteria strain, relative to a level of the nitrogen fixation produced by the live microbe or the reporter bacteria strain prior to the contacting, as determined by an in vitro assay comprising:
(i) incubating the live microbe or the reporter bacteria strain aerobically in nitrogen-free media at an OD₆₀₀ of 0.02, wherein the live microbe or the reporter bacteria strain is transformed with a luciferase reporter plasmid configured to produce a higher level luminescence in response to nitrogen fixation;
(ii) contacting the live microbe or the reporter bacteria strain with luciferin 24 hours after the incubating; and
(iii) quantifying the level of the luminescence using a luminometer, wherein a higher level of luminescence corresponds to a higher degree of nitrogen fixation by the live microbe or the reporter bacteria strain; or
(c) a combination of (a) and (b).

2. (canceled)

3. (canceled)

4. (canceled)

5. The liquid composition of claim 1, wherein the excipient, diluent, or carrier is water.

6. (canceled)

7. (canceled)

8. The liquid composition of claim 1, wherein the compound or salt thereof is of Formula Ia, Ib, Ic, or Id: 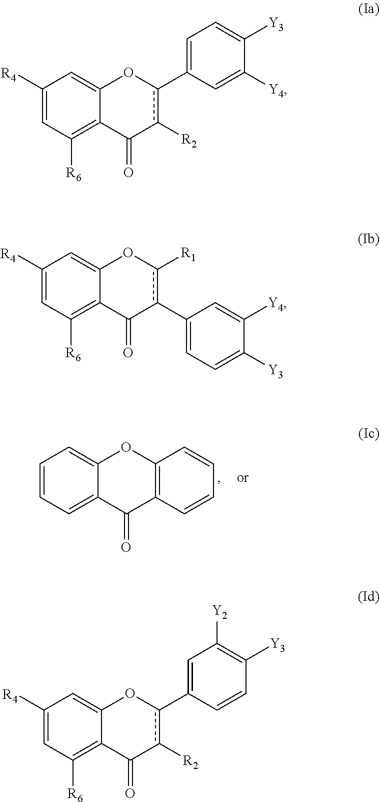
wherein R₁, R₂, R₄, R₆, Y₂, Y₃, and Y₄ are as defined in claim 1.

9. The liquid composition of claim 8, wherein the compound or salt thereof is of Formula Ia and is selected from the group consisting of: 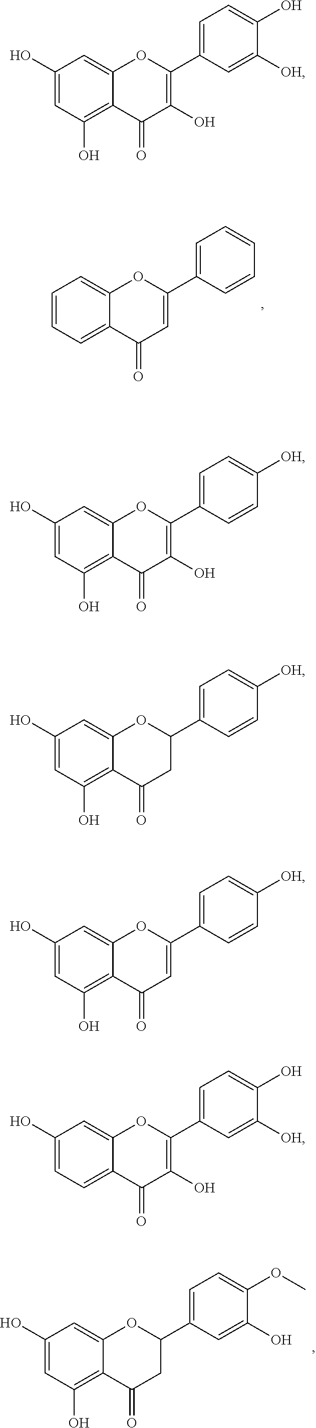
or a salt of any of these.

10. (canceled)

11. (canceled)

12. The liquid composition of claim 8, wherein the compound or a salt thereof is of Formula Id having a structure of:

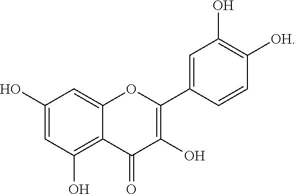 [SMILES: O=c1c(O)c(-c2ccc(O)c(O)c2)oc2cc(O)cc(O)c12]

13. (canceled)

14. (canceled)

15. (canceled)

16. (canceled)

17. The liquid composition of claim 1, wherein the live microbe is present in soil.

18. The liquid composition of claim 17, wherein the live microbe is a bacteria strain, an actinomycete, a fungus, a protozoa, or any combination thereof.

19. The liquid composition of claim 18, wherein the live microbe is a bacteria strain of genus Bacillus, Azobacter, Pseudomonas, Nitrobacter, Clostrodium, or any combination thereof.

20. The liquid composition of claim 17, wherein the live microbe is selected from the group consisting of: Azotobacter chroococcum, Pseudomonas stutzeri, Pseudomonas pseudoalcaligenes, Massilia tieshanesis, Massilia aerilata, Massilia putida, Bacillus solisilvae, Bacillus niacini, Massilia agilis, Bacillus wiedmannii, Massilia brevitalea, Bacillus acidiceler, Bacillus toyonensis, Pseudomonas otitidis, Pseudomonas citronellolis, Paenibacillus qinlingensis, Massilia solisilvae, Massilia terrae, Bacillus paramycoides, Massilia aurea, Bacillus acidicola, Panenibacillus alginolyticus, Bacillus novalis, Pseudomonas aeruginosa, Bacillus halmapalus, Pseudomonas knackmussii, Klebsiella pneumoniae, Klebsiella variicola, Klebsiella oxytoca, Pseudomonas aeruginosa, Serratia marcescens, Bacillus amyloliquefaciens, Gluconacetobacter diazotrophicus Massilia arvi, Massilia agri, Massilia pinisoli, Bacillus megaterium, Bacillus bataviensis, Massilia chloroacetimidivorans, Bacillus mycoides, Bacillus flexus, Bacillus simplex, Pseudomonas balearica, Pseudomonas plecoglossicida, Caballeronia turbans, Psychobacillus lasiicaptis, Bacillus soli, Bacillus cohnii, Cupriavidus campinensis, Brevibacterium frigoritolerans, Bacillus pocheonensis, Pseudomonas monteilii, Bacillus vireti, Bacillus pacificus, Paenibacillus taihuensis, Azotobacter beijerinckii, Paenibacillus contaminans, Bacillus drentensis, Bacillus thuringiensis, Bacillus firmus, Bacillus cereus, Bacillus mobilis, Bacillus luciferensis, Massilia niastensis, Bacillus cucumis, Pseudomonas flavescens, Massilia timonae, Massilia kyonggiensis, Pseudomonas indica, Bacillus phyllosphaerae, Pseudomonas guguanensis, Paenibacillus beijingensis, Bacillus pseudomycoides, Adhaeribacter terreus, Microvirga zambiensis, Pseudomonas oryzae, or any combination thereof.

21. A method comprising contacting a composition with a live microbe, wherein the composition comprises:
(a) a compound or salt thereof of Formula I: 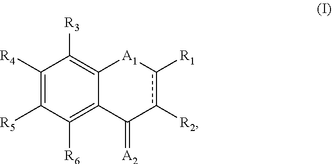
wherein:
A¹ and A² are independently O or S;
R₁ and R₂ are independently —H, —OH, —COOH, —SH, C₁-C₆ alkyl, C₃-C₆ cycloalkyl, or —Xₚ, wherein —Xₚ is: 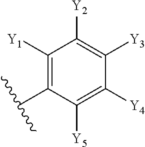
wherein Y₁, Y₂, Y₃, Y₄, and Y₅ are independently —H, —OH, —SH, —F, —Cl, —Br, —I, or —O—Z₁, wherein Z₁ is C₁-C₄ alkyl; or
wherein R₁ and R₂ along with the carbon atoms connecting them form a five or six-membered cycloalkyl ring or cycloalkenyl ring, or a five or six-membered aryl ring; and
R₃, R₄, R₅, and R₆ are independently —H, —OH, —F, —Cl, —Br, —I, or —SH; and
(b) an excipient, diluent, or carrier;
wherein the contacting is sufficient to produce:
(a) an increased level of soluble orthophosphate of at least about 20% after contacting the amount of the compound or salt thereof with a live microbe, optionally a live Bacillus megaterium bacteria strain, relative to a level of the soluble orthophosphate produced by the live microbe prior to the contacting, as determined by an in vitro assay comprising:
(i) incubating the live microbe at an optical density at 600 nm (OD₆₀₀) of 0.02 with tricalcium phosphate at a final concentration of about 50 mM;
(ii) collecting a sample of a liquid culture from the live microbe 72 hours after the incubating; and
(iii) quantifying the level of the orthophosphate in the liquid culture using a malachite-green method; or
(b) an increased level of nitrogen fixation after contacting the amount of the compound or salt thereof with the live microbe or a reporter bacteria strain, optionally a reporter Azotobacter vinelandii bacteria strain, relative to a level of the nitrogen fixation produced by the live microbe or the reporter bacteria strain prior to the contacting, as determined by an in vitro assay comprising:
(i) incubating the live microbe or the reporter bacteria strain aerobically in nitrogen-free media at an OD₆₀₀ of 0.02, wherein the live microbe or the reporter bacteria strain is transformed with a luciferase reporter plasmid configured to produce a higher level luminescence in response to nitrogen fixation;
(ii) contacting the live microbe or the reporter bacteria strain with luciferin 24 hours after the incubating; and
(iii) quantifying the level of the luminescence using a luminometer, wherein a higher level of luminescence corresponds to a higher degree of nitrogen fixation by the live microbe or the reporter bacteria strain; or
(c) a combination of (a) and (b).

22. The method of claim 21, wherein the excipient, diluent, or carrier is water.

23. (canceled)

24. (canceled)

25. The method of claim 21, wherein the compound or salt thereof is of Formula Ia, Ib, Ic, or Id: 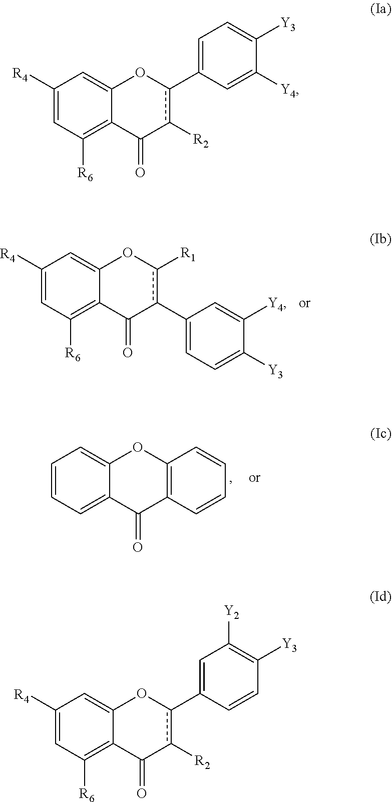
wherein R₁, R₂, R₄, R₆, Y₂, Y₃, and Y₄ are as defined in claim 21.

26. The method of claim 25, wherein the compound or salt thereof is of Formula Ia and is selected from the group consisting of: 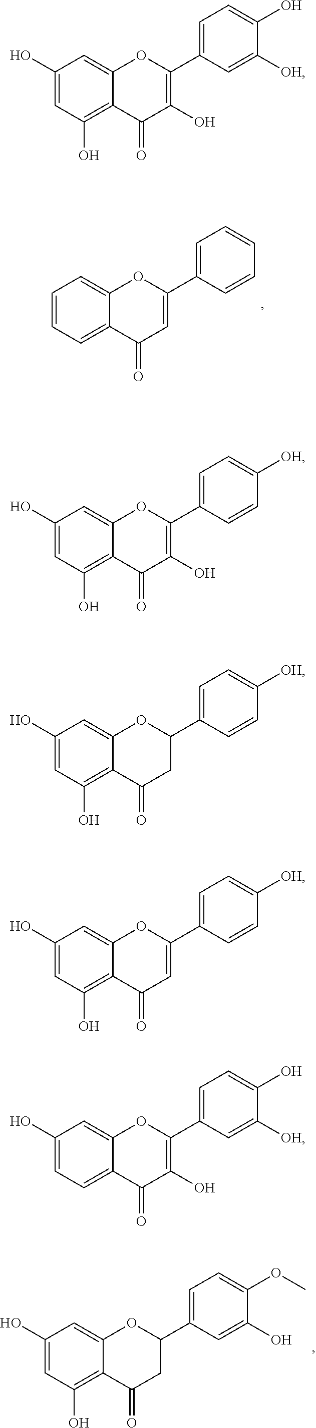
or a salt of any of these.

27. The method of claim 25, wherein the compound or salt thereof is of Formula Ib and is selected from the group consisting of: 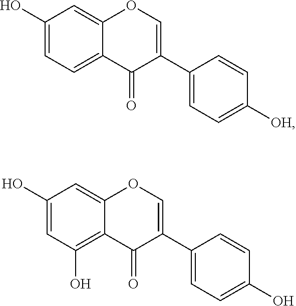 [SMILES: O=c1c(-c2ccc(O)cc2)coc2cc(O)ccc12 ; O=c1c(-c2ccc(O)cc2)coc2cc(O)cc(O)c12]
or a salt of any of these.

28. The method of claim 25, wherein the compound or salt thereof is of Formula Ic or a salt thereof.

29. The method of claim 25, wherein the compound of Formula Id is: 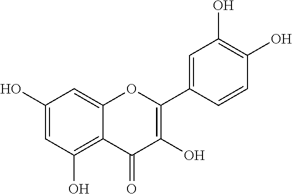 [SMILES: O=c1c(O)c(-c2ccc(O)c(O)c2)oc2cc(O)cc(O)c12]
or a salt thereof.

30. (canceled)

31. (canceled)

32. (canceled)

33. (canceled)

34. The method of claim 21, wherein the live microbe is present in soil.

35. The method of claim 21, wherein the live microbe is a bacteria strain, an actinomycete, a fungus, a protozoa, or any combination thereof.

36. The method of claim 35, wherein the live microbe is a bacteria strain of genus Bacillus, Azobacter, Pseudomonas, Nitrobacter, Clostrodium, or any combination thereof.

37. The method of claim 35, wherein the live microbe is selected from the group consisting of: Azotobacter chroococcum, Pseudomonas stutzeri, Pseudomonas pseudoalcaligenes, Massilia tieshanesis, Massilia aerilata, Massilia putida, Bacillus solisilvae, Bacillus niacini, Massilia agilis, Bacillus wiedmannii, Massilia brevitalea, Bacillus acidiceler, Bacillus toyonensis, Pseudomonas otitidis, Pseudomonas citronellolis, Paenibacillus qinlingensis, Massilia solisilvae, Massilia terrae, Bacillus paramycoides, Massilia aurea, Bacillus acidicola, Panenibacillus alginolyticus, Bacillus novalis, Pseudomonas aeruginosa, Bacillus halmapalus, Pseudomonas knackmussii, Klebsiella pneumoniae, Klebsiella variicola, Klebsiella oxytoca, Pseudomonas aeruginosa, Serratia marcescens, Bacillus amyloliquefaciens, Gluconacetobacter diazotrophicus Massilia arvi, Massilia agri, Massilia pinisoli, Bacillus megaterium, Bacillus bataviensis, Massilia chloroacetimidivorans, Bacillus mycoides, Bacillus flexus, Bacillus simplex, Pseudomonas balearica, Pseudomonas plecoglossicida, Caballeronia turbans, Psychobacillus lasiicaptis, Bacillus soli, Bacillus cohnii, Cupriavidus campinensis, Brevibacterium frigoritolerans, Bacillus pocheonensis, Pseudomonas monteilii, Bacillus vireti, Bacillus pacificus, Paenibacillus taihuensis, Azotobacter beijerinckii, Paenibacillus contaminans, Bacillus drentensis, Bacillus thuringiensis, Bacillus firmus, Bacillus cereus, Bacillus mobilis, Bacillus luciferensis, Massilia niastensis, Bacillus cucumis, Pseudomonas flavescens, Massilia timonae, Massilia kyonggiensis, Pseudomonas indica, Bacillus phyllosphaerae, Pseudomonas guguanensis, Paenibacillus beijingensis, Bacillus pseudomycoides, Adhaeribacter terreus, Microvirga zambiensis, Pseudomonas oryzae, or any combination thereof.

38. (canceled)

39. (canceled)

40. A method of improving health of a plant, comprising contacting a plant present in soil comprising a live microbe with the liquid composition of claim 1, wherein the contacting is sufficient to increase a biomass of the plant or an amount of greenness of the plant, relative to a biomass or amount of greenness of a comparable plant grown for a comparable amount of time and not contacted with the composition, thereby improving the health of the plant.

41. The method of claim 40, wherein the contacting comprises at least one of contacting a leaf of the plant, contacting a stem of the plant, or contacting a root of the plant.

42. (canceled)

43. (canceled)

44. The method of claim 40, wherein the contacting substantially maintains an amount of greenness of the plant for a longer period of time, relative to an amount of greenness of the comparable plant.

45. A method of making a plant, comprising:
(a) contacting a plant seed with an exogenous compound or salt thereof of Formula I, 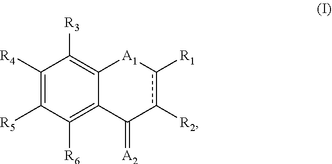
wherein:
A¹ and A² are independently O or S;
R₁ and R₂ are independently —H, —OH, —COOH, —SH, C₁-C₆ alkyl, C₃-C₆ cycloalkyl, or —Xₚ, wherein —Xₚ is: 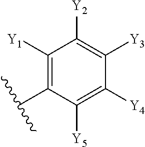
wherein Y₁, Y₂, Y₃, Y₄, and Y₅ are independently —H, —OH, —SH, —F, —Cl, —Br, —I, or —O—Z₁, wherein Z₁ is C₁-C₄ alkyl; or
wherein R₁ and R₂ along with the carbon atoms connecting them form a five or six-membered cycloalkyl ring or cycloalkenyl ring, or a five or six-membered aryl ring; and
R₃, R₄, R₅, and R₆ are independently —H, —OH, —F, —Cl, —Br, —I, or —SH; and
(b) planting the plant seed into soil comprising a live microbe, thereby making a plant.

46. The method of claim 45, wherein the contacting is sufficient to increase a biomass of the plant, relative to a biomass of a comparable plant produced from a seed not contacted with the composition and grown for a comparable time.

47. The method of claim 45, wherein the contacting is sufficient to increase an amount of greenness of the plant, relative to an amount of greenness of a comparable plant produced from a seed not contacted with the composition and grown for a comparable time.

48. The method of claim 45, wherein the compound or salt thereof is of Formula Ia, Ib, Ic, or Id: 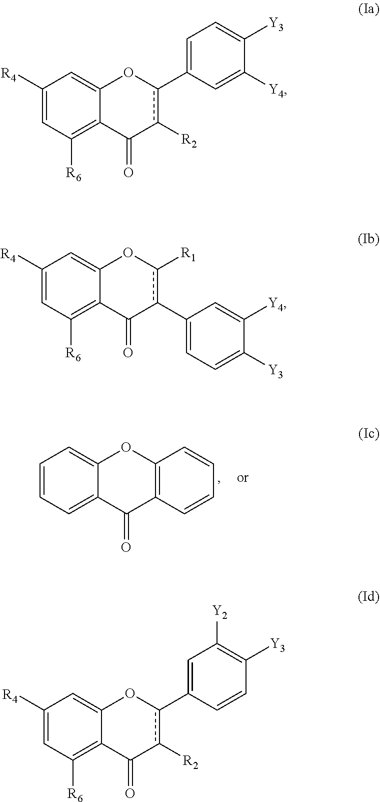
wherein R₁, R₂, R₄, R₆, Y₂, Y₃, and Y₄ are as defined in claim 45.

49. The method of claim 48, wherein the compound or salt thereof is of Formula Ia and is selected from the group consisting of: 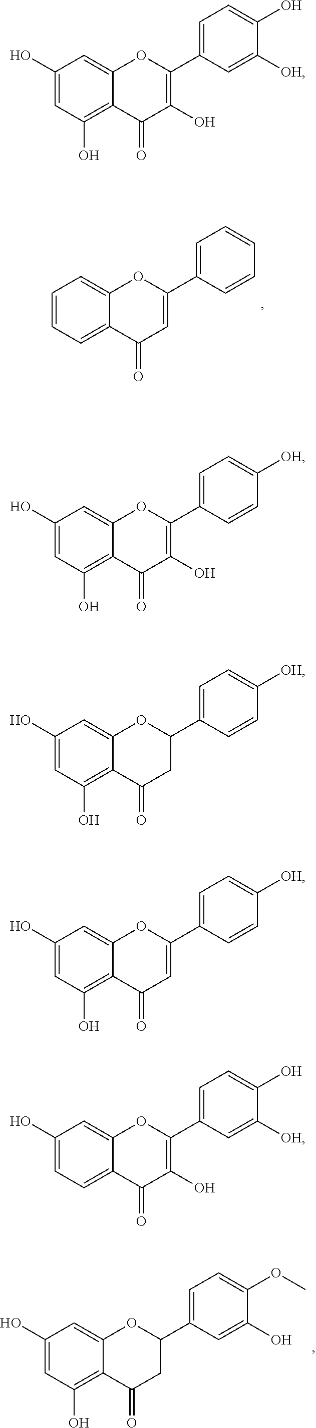
or a salt of any of these.

50. The method of claim 48, wherein the compound or salt thereof is of Formula Ib and is selected from the group consisting of: 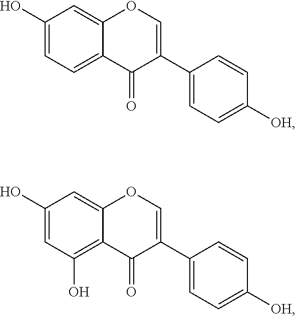 [SMILES: O=c1c(-c2ccc(O)cc2)coc2cc(O)ccc12 ; O=c1c(-c2ccc(O)cc2)coc2cc(O)cc(O)c12]
or a salt of any of these.

51. The method of claim 48, wherein the compound or salt thereof is of Formula Ic or a salt thereof.

52. The method of claim 48, wherein the compound or a salt thereof is of Formula Id having the structure:

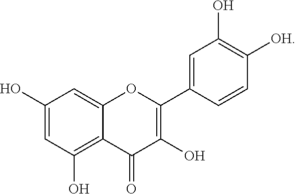 [SMILES: O=c1c(O)c(-c2ccc(O)c(O)c2)oc2cc(O)cc(O)c12]

53. (canceled)

54. (canceled)

55. (canceled)

56. (canceled)

57. (canceled)

58. (canceled)

59. (canceled)

60. (canceled)

61. (canceled)

62. (canceled)

63. The liquid composition of claim 1, wherein the liquid composition is a suspension concentrate.

64. The liquid composition of claim 1, wherein the compound or its salt is present at a concentration of from about 10-30% w/w.

65. The method of claim 21, wherein the composition is a suspension concentrate.

66. The method of claim 21, wherein the compound or its salt is present at a concentration of from about 10-30% w/w.

67. The method of claim 45, wherein the exogenous compound is in the form of a suspension concentrate.

68. The method of claim 67, wherein the suspension concentrate comprises an excipient, diluent, or carrier.

69. The method of claim 68, wherein the excipient, diluent, or carrier is water

70. The method of claim 45, wherein the compound or its salt is present in the suspension concentrate at a concentration of from about 10-30% w/w.
